(19) United States
(12) Patent Application Publication
(10) Pub. No.: US 2009/0048672 A1
(43) Pub. Date: Feb. 19, 2009

(54) BONE SUPPORT DEVICES AND METHODS

(75) Inventor: Kirk Essenmacher, Palo Alto, CA (US)

Correspondence Address: WILSON SONSINI GOODRICH & ROSATI, 650 PAGE MILL ROAD, PALO ALTO, CA, 94304-1050, US

(73) Assignee: JUVENTAS, INC., Palo Alto, CA (US)

(21) Appl. No.: 12/141,775

(22) Filed: Jun. 18, 2008

Related U.S. Application Data
(60) Provisional application No. 60/945,007, filed on Jun. 19, 2007.

(51) Int. Cl. A61F 2/02; A61F 2/08; A61B 17/56

(52) U.S. Cl. 623/11.11; 623/14.12; 606/62

(57) ABSTRACT

Provided herein are methods, devices and kits for reducing the risk of fracturing a bone. A device is provided comprising an implantable pad for absorbing, deflecting, and/or diffusing an exterior impact force on a bone. A device is provided for strengthening and/or reinforcing a bone at typical fracture sites prior to fracture and/or along lines of tension and compression forces. The devices may comprise a coil adapted to be implanted in the bone. The device may comprise a rod adapted to be implanted in the bone. The devices may comprise a deployable element adapted to be deployed in the bone. The device may comprise a scaffold adapted to be implanted in the bone. The device may comprise a reinforcing material and/or means for reinforcing and/or strengthening the bone at typical fracture sites prior to fracture and/or along lines of tension and compression forces.

### CROSS-REFERENCE

This application claims the benefit of U.S. Provisional Application No. 60/945,007, filed Jun. 19, 2007, which application is incorporated in its entirety herein by reference.

### BACKGROUND OF THE INVENTION

Osteoporosis is characterized by low bone mass and structural deterioration of bone tissue, leading to bone fragility and an increased susceptibility to bone fractures, especially of the hip bones, spine, and wrist bones. Osteoporosis occurs primarily as a result of normal aging, but can arise as a result of impaired development of peak bone mass or excessive bone loss during adulthood. The National Osteoporosis Foundation estimated that one in four men and one in two women and over the age of 50 will have an osteoporosis-related fracture in his/her remaining lifetime. The number of fractures is expected to grow as the aging population grows. The World Health Organization (WHO) estimated in 2003 that the number of osteoporosis fractures would double over the next fifty years.

Most of the osteoporosis-related hip fractures are due to falls. Hip fractures often result in hospitalization, and complete recovery is difficult—if it is ever achieved. Many patients require at least a year of nursing home care. Many patients require long term nursing home care, and many are permanently disabled following a hip fracture. Too often, osteoporotic adults who suffer a fall and hip fracture die within one year.

Osteoporotic fractures costs are great. The estimated national direct care expenditures (including hospitals, nursing homes, and outpatient services) for osteoporotic fractures were about $18 billion dollars per year in 2002. Hip fracture, in particular, usually requires prompt surgery, and involves serious risks such as deep vein thrombosis and a pulmonary embolism. The WHO postulated that osteoporosis results in more hospital bed days than stroke, myocardial infarction or breast cancer. The treatment after fracture frequently is hip replacement and/or fracture repair.

Women can lose bone mass in the five to seven years following menopause, making them more susceptible to osteoporosis. There are many preventative measures recommended for adults (male or female) to reduce the rate of bone loss associated with osteoporosis and to reduce the risk of pathologic fractures, including weight bearing exercise, nutritional supplements, and medication.

Weight bearing exercise puts stress on bones, which stimulates bone production, thus strengthening bones. Nutritional supplements, calcium and Vitamin D in particular, are recommended to help build healthy bones.

Medications such as biophoshpanates help regulate calcium and slow or even prevent bone breakdown. Since bone replacement is a normal process, biophoshpanates slow the rate of breakdown of old bone (inhibiting osteoclasts responsible for bone breakdown) while allowing bone replacement (osteoblasts) to continue. Calcitonin is a naturally occurring hormone that also inhibits the function of osteoclasts. Both injectable calcitonin and nasal-administered Calcitonin are available to inhibit osteoclast function.

Another treatment suited to help women following menopause by increasing bone mass is estrogen therapy. Multiple studies have shown benefits to estrogen therapy, including increased bone mass, lower risk of osteoporosis and fractured bones, lower cholesterol, decreased risk of colon cancer. However, some studies have led to fear of developing blood clots, stroke, increase in uterine cancer, and increased risk of breast cancer and, thus, estrogen therapy may be disfavored as a treatment regimen for osteoporosis.

Selective Estrogen Receptor Modulators (SERMs) are another therapy that provides some of the benefits of estrogen replacement by increasing bone mass and reducing cholesterol. Other new medications are also being studied to reduce the rate of bone loss.

### SUMMARY OF THE INVENTION

Nonetheless, there remains a need for methods, devices, and systems to prevent and treat bone fracture.

Provided herein is a device comprising an implantable pad. In some embodiments, the pad is adapted to absorb an exterior impact force on a bone, deflect an exterior impact force on a bone, diffuse an exterior impact force on a bone, strengthen a bone, and/or reinforce a bone.

Provided herein is a device comprising a coil having a portion adapted to be implanted in a bone. In some embodiments, the device further comprises an implantable pad adapted to interface with the bone. In some embodiments, the pad and the coil are adapted to cooperate in absorbing an exterior impact force on a bone, deflecting an exterior impact force on a bone, diffusing an exterior impact force on a bone, strengthening a bone, and/or reinforcing a bone.

The coil may be adapted to substantially align with at least one direction of load in the bone experienced in a bone during a fall. The coil may be adapted to substantially align with at least one direction of load in the bone experienced in normal use. Normal use may include, for non-limiting example, at least one of walking, lifting, bending, sitting, running, leaning, and standing.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising a rod having a portion adapted to be implanted in a bone. In some embodiments, the device further comprises an implantable pad adapted to interface with the bone. The pad and the rod may be adapted to cooperate in absorbing an exterior impact force on a bone, deflecting an exterior impact force on a bone, diffusing an exterior impact force on a bone, strengthening a bone, and/or reinforcing a bone.

The rod may be adapted to substantially align with at least one direction of load in the bone experienced in a bone during a fall. In some embodiments, the rod is adapted to substantially align with at least one direction of load in the bone experienced in normal use. Normal use may include, for non-limiting example, at least one of walking, lifting, bending, sitting, running, leaning, and standing.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising a multiple-rod scaffold adapted to be implanted within a bone. The scaffold may comprise a plurality of rods. The scaffold may comprise a plurality of wires. The scaffold may comprise a plurality of coils. The scaffold may comprise any combination of rods, wires, and/or coils. In some embodiments, at least one of the rods, wires, and coils of the scaffold is adapted to be implanted substantially parallel relative to another rod, wire, or coil of the scaffold. At least one of the rods, wires, and/or coils is adapted to be implanted at a displaced position relative to another rod, wire, and/or coil of the scaffold.

At least one of the rods, coils, and/or wires may be adjustable in length.

In some embodiments, the scaffold is adapted to substantially align with at least one direction of load in the bone experienced in a bone during a fall. The scaffold may be adapted to substantially align with at least one direction of load in the bone experienced in normal use. Normal use may include, for non-limiting example, at least one of walking, lifting, bending, sitting, running, leaning, and standing. The scaffold may be capable of absorbing an exterior impact force on a bone, deflecting an exterior impact force on a bone, diffusing an exterior impact force on a bone, strengthening a bone, and/or reinforcing a bone.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising an implant adapted to be implanted in a bone at a first orientation and a first position relative to the bone.

The device may comprise a deployable element adapted to deploy in the bone at a second orientation and second position relative to the bone. The deployable element may be coupled to the implant. The deployable element may unfold, or fold. The deployable element may be adapted to deploy in the bone. The deployable element may expand radially from the implant, and/or expand distally from the implant.

The deployable element may deploy within the femur head. The deployable element may be a generally ring-shaped support once deployed within the bone. The generally ring-shaped support may generally abut cortical bone of the bone, or be implanted where spongy bone interfaces with cortical bone.

The deployable element may be implanted into a cavity in the bone. The cavity may be pre-formed prior to implantation of the support, or be formed by the generally ring-shaped support. The cavity may exist prior to implantation of the implant.

In an embodiment wherein the bone is a femur, the deployed deployable element may be a generally ring-shaped support within at least one of the femur head, the femur neck, and/or anywhere along the axis defined by the femur head, femur neck and femur body. The deployed deployable element may be a cylinder-like support within at least one of the femur head, the femur neck, or anywhere along the axis defined by the femur head, femur neck and femur body.

At least one of the implant and the deployable element of the implant may be adapted to substantially align with at least one direction of load in the bone experienced in a bone during a fall. At least one of the implant and the deployable element of the implant may be adapted to substantially align with at least one direction of load in the bone experienced in normal use. Normal use may include, for non-limiting example, at least one of walking, lifting, bending, sitting, running, leaning, and standing. In some embodiments, at least one of the implant and the deployable element is capable of absorbing an exterior impact force on a bone, deflecting an exterior impact force on a bone, diffusing an exterior impact force on a bone, strengthening a bone, and/or reinforcing a bone.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple. In some embodiments, the generally ring-shaped support is also the fastening element, comprised of least one of bone cement, an elastomer, a glue, and a spongy foam. In some embodiments, the generally ring-shaped support comprises a fastening element which may comprise at least one of a corkscrew fixating element, a nail, and a staple.

Provided herein is a device comprising an means for maintaining in position an means for absorbing a force on a bone. In some embodiments the force results from a fall. In some embodiments, the force results from normal use. Normal use may include, for non-limiting example, at least one of walking, lifting, bending, sitting, running, leaning, and standing. The means for absorbing may be implantable. The means for maintaining in position may be implantable.

The means for absorbing may comprise at least one of a pad, a rod, a collapsible rod, and a coil. The means for absorbing may comprise at least one of an elastomer, a silicone, a memory metal, and spongy foam. The means for absorbing may comprise a material having a higher yield strength than bone. The means for absorbing may comprise a material having about the same yield strength as trabecular bone. The means for absorbing may comprise a material having about the same yield strength as cortical bone. The means for absorbing may comprise at least one of metal and bone cement.

The means for maintaining in position may comprise a fastening element capable of fastening a distal end of the means for absorbing to spongy bone and/or trabecular bone, and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising an implantable means for maintaining in position a means for deflecting a force on a bone. In some embodiments the force results from a fall. In some embodiments, the force results from normal use. Normal use may include, for non-limiting example, at least one of walking, lifting, bending, sitting, running, leaning, and standing.

In some embodiments, the means for deflecting the force on the bone deflects the force in a direction whereby the force is directed to at least one of a portion of the bone that can withstand the force without fracturing the bone, another body part without fracturing the bone, and/or a material external to the body without fracturing the bone. The means for deflecting the force on the bone may deflect the force to at least one of a portion of the bone that is sufficiently strong to withstand the force without fracturing the bone, and a portion of a body that is sufficiently strong to withstand the force without fracturing the bone and without fracturing the portion of the body.

In some embodiments, the device comprises a reinforcing element capable of reinforcing the portion of the bone, wherein the reinforcing element comprises at least one of bone cement, silicone, metal, including memory metal, and elastomers. In some embodiments, the device comprises a reinforcing element capable of reinforcing the portion of the body, wherein the reinforcing element comprises at least one of bone cement, silicone, metal, including memory metal, and elastomers.

In some embodiments, the means for maintaining in position comprises a fastening element capable of fastening a distal end of the means for deflecting to spongy bone and/or trabecular bone, and/or cortical bone of the bone. In some embodiments, the fastening element comprises at least one of bone cement, an elastomer, a glue, and a spongy foam. The fastening element may comprise at least one of a corkscrew fixating element, a nail, and a staple.

Provided herein is a device comprising an means for maintaining in position a means for diffusing a force on a bone. The means for maintaining in position may be implantable. The means for diffusing a force on a bone may be implantable. In some embodiments the force results from a fall. In some embodiments, the force results from normal use. Normal use may include, for non-limiting example, at least one of walking, lifting, bending, sitting, running, leaning, and standing.

In some embodiments, the means for maintaining in position comprises a fastening element capable of fastening a distal end of the means for deflecting to spongy bone and/or trabecular bone, and/or cortical bone of the bone. In some embodiments, the fastening element comprises at least one of bone cement, an elastomer, a glue, and a spongy foam. The fastening element may comprise at least one of a corkscrew fixating element, a nail, and a staple.

In some embodiments described herein comprising an implantable pad, at least a portion of the pad is adapted to be implanted inside of the bone. In some embodiments, the pad is implantable within a spongy bone portion of the bone. In some embodiments, the pad is adapted to be attached to a compact bone portion of the bone. In some embodiments, at least a portion of the pad is adapted to be implanted outside of the bone. In some embodiments, the pad is adapted to be attached to the bone. In some embodiments the pad is adapted to slide along a surface of an exterior portion of the bone.

In some embodiments of the devices provided herein, the device further comprises reinforcing bone cement. The cement may be adapted to be implanted in a location in the bone of high stress and/or a location in the bone of high strain during a fall. The cement may be adapted to be implanted in a location in the bone of high stress and/or a location in the bone of high strain during a normal use. Normal use may include, for non-limiting example, at least one of walking, lifting, bending, sitting, running, leaning, and standing.

Provided herein is a device comprising an implantable means for maintaining in position a means for reinforcing a bone, wherein the means for reinforcing is capable of reinforcing the bone to withstand a force on the bone. In some embodiments, the means for reinforcing is adapted to be implanted in a location in the bone of high stress and/or a location in the bone of high strain during a fall. In some embodiments, the means for reinforcing is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for reinforcing is adapted to be implanted in a location in the bone of high stress and/or a location in the bone of high strain during a fall. In some embodiments, the means for reinforcing is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for reinforcing comprises at least one of a pad, a rod, a collapsible rod, and a coil. In some embodiments, the means for reinforcing comprises at least one of an elastomer, a silicone, a memory metal, and spongy foam. The means for reinforcing may comprise a material having a higher yield strength than bone. The means for reinforcing may comprise a material having about the same yield strength as trabecular bone. In some embodiments, the means for reinforcing comprises a material having about the same yield strength as cortical bone. In some embodiments, the means for reinforcing comprises at least one of metal and bone cement.

In some embodiments, the means for maintaining in position comprises a fastening element capable of fastening a distal end of the means for reinforcing to spongy bone and/or trabecular bone, and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising an implantable means for maintaining in position a means for strengthening a bone, wherein the means for strengthening is capable of strengthening the bone to withstand a force on the bone. In some embodiments, the means for strengthening is adapted to be implanted in a location in the bone of high stress and/or a location in the bone of high strain during a fall. In some embodiments, the means for strengthening is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for strengthening comprises at least one of a pad, a rod, a collapsible rod, and a coil. In some embodiments, the means for strengthening comprises at least one of an elastomer, a silicone, a memory metal, and spongy foam. The means for strengthening may comprise a material having a higher yield strength than bone. The means for strengthening may comprise a material having about the same yield strength as trabecular bone. In some embodiments, the means for strengthening comprises a material having about the same yield strength as cortical bone. In some embodiments, the means for strengthening comprises at least one of metal and bone cement.

In some embodiments, the means for maintaining in position comprises a fastening element capable of fastening a distal end of the means for strengthening to spongy bone and/or trabecular bone, and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a method for reducing the risk of fracturing a bone of a patient comprising implanting a pad adapted to interface with a bone. Provided herein is a method for reducing the risk of fracturing a bone of a patient comprising implanting a device comprising a pad adapted to interface with a bone. The pad may be any pad as described herein. The device may be any device comprising a pad as described herein. The pad may be adapted to absorb an exterior impact force on a bone, deflect an exterior impact force on a bone, diffuse an exterior impact force on a bone, strengthen a bone, and/or reinforce a bone.

Provided herein is a method for reducing the risk of fracturing a bone of a patient comprising delivering a portion of a coil within a bone. Provided herein is a method for reducing the risk of fracturing a bone of a patient comprising delivering a portion of a device comprising a coil within a bone. Any coil as described herein may be used in the embodiments of the method. Any device comprising a coil as described herein may be used in the embodiments of the method. The method may further comprise implanting a pad adapted to interface with the bone. The pad may be any pad as described herein. The pad may be adapted to absorb an exterior impact force on a bone, deflect an exterior impact force on a bone, diffuse an exterior impact force on a bone, strengthen a bone, and/or reinforce a bone.

Provided herein is a method for reducing the risk of fracturing a bone of a patient comprising delivering a portion of a rod within a bone. Provided herein is a method for reducing the risk of fracturing a bone of a patient comprising delivering a portion of a device comprising a rod within a bone. Rods and devices comprising a rod described herein may be used in the methods provided herein. The method may further comprise implanting a pad adapted to interface with the bone. The pad may be any pad as described herein. The pad may be adapted to absorb, deflect, and/or diffuse an exterior impact force on a bone. The pad and the rod may be adapted to cooperate in absorbing an exterior impact force on a bone, deflecting an exterior impact force on a bone, diffusing an exterior impact force on a bone, strengthening a bone, and/or reinforcing a bone.

Provided herein is a method for reducing the risk of fracturing a bone of a patient comprising implanting a multiple-rod scaffold within a bone. Provided herein is a method for reducing the risk of fracturing a bone of a patient comprising implanting a device comprising a multiple-rod scaffold within a bone. Multiple-rod scaffolds and devices comprising a multiple-rod scaffold as described herein may be used in the methods provided herein. The implanting may comprise orienting each rod substantially parallel to each other rod, and positioning each rod at a displaced position in the bone relative to each other rod.

Provided herein is a method for reducing the risk of fracturing a bone of a patient comprising implanting a device in a bone at a first orientation and a first position relative to the bone, wherein the device comprises an implant and the implant comprises a deployable element adapted to deploy within the bone at a second orientation and second position relative to the bone. Provided herein is a method for reducing the risk of fracturing a bone of a patient comprising implanting an implant in a bone at a first orientation and a first position relative to the bone, wherein the implant comprises a deployable element adapted to deploy within the bone at a second orientation and second position relative to the bone. Devices comprising implants having deployable elements and implants having deployable elements as described herein may be used in embodiments of the method. The method may further comprise deploying the deployable element at the second orientation and second position relative to the bone.

Provided herein is a method for reducing the risk of fracturing a bone of a patient comprising implanting a means for absorbing force on a bone. Provided herein is a method for reducing the risk of fracturing a bone of a patient comprising implanting a device comprising a means for absorbing force on a bone. In some embodiments of the methods, the force results from a fall. In some embodiments of the methods, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for absorbing force on a bone as described herein, and means for absorbing force on a bone as described herein may be used with embodiments of the method. The method may further comprise securing the means for absorbing force within tissue. Means for securing the means for absorbing force as described herein may be used in embodiments of the method. Devices comprising means for securing the means for absorbing force as described herein may be used in embodiments of the method.

Provided herein is a method for reducing the risk of fracturing a bone of a patient comprising implanting a means for deflecting force on a bone. Provided herein is a method for reducing the risk of fracturing a bone of a patient comprising implanting a device comprising a means for deflecting force on a bone. In some embodiments of the methods, the force results from a fall. In some embodiments of the methods, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for deflecting force on a bone as described herein, and means for deflecting force on a bone as described herein may be used with embodiments of the method. The method may further comprise securing the means for deflecting force within tissue. Means for securing the means for deflecting force as described herein may be used in embodiments of the method. Devices comprising means for securing the means for deflecting force as described herein may be used in embodiments of the method.

Provided herein is a method for reducing the risk of fracturing a bone of a patient comprising implanting a means for diffusing force on a bone. Provided herein is a method for reducing the risk of fracturing a bone of a patient comprising implanting a device comprising a means for diffusing force on a bone. In some embodiments of the methods, the force results from a fall. In some embodiments, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for diffusing force on a bone as described herein, and means for diffusing force on a bone as described herein may be used with embodiments of the method. The method may further comprise securing the means for diffusing force within tissue. Means for securing the means for diffusing force as described herein may be used in embodiments of the method. Devices comprising means for securing the means for diffusing force as described herein may be used in embodiments of the method.

Provided herein is a method for reducing the risk of fracturing a bone of a patient comprising implanting a means for reinforcing a bone, and securing the means for reinforcing within tissue. In some embodiments, the means for reinforcing is capable of reinforcing the bone to withstand a force. In some embodiments, the bone is osteoporotic. In some embodiments, the force is from normal loading of the bone. Normal loading may comprise, for non-limiting example, at least one of walking, lifting, bending, sitting, running, leaning, and standing. In some embodiments, the force results from a fall. Devices comprising means for reinforcing a bone as described herein may be used with embodiments of the method. Devices comprising means for securing the means for reinforcing force as described herein may be used in embodiments of the method.

In some embodiments, the means for reinforcing is adapted to be placed in a femur at a typical fracture region comprising at least one of the subcapital region of the femur, the cervical region of the femur, the basal region of the femur, and the pertrochaneric region of the femur. Some embodiments of the method comprise placing the means for reinforcing in a femur at a typical fracture region, wherein the typical fracture region comprises at least one of the subcapital region of the femur, the cervical region of the femur, the basal region of the femur, and the pertrochaneric region of the femur.

Provided herein is a method for reducing the risk of fracturing a bone of a patient comprising implanting a means for strengthening a bone, and securing the means for strengthening within tissue. In some embodiments, the means for strengthening is capable of strengthening the bone to withstand a force. In some embodiments, the bone is osteoporotic. In some embodiments, the force is from normal loading of the bone. Normal loading may comprise, for non-limiting example, at least one of walking, lifting, bending, sitting, running, leaning, and standing. In some embodiments the force is from a fall. In some embodiments, the force results from a fall. Devices comprising means for strengthening a bone as described herein may be used with embodiments of the method. Devices comprising means for securing the means for strengthening force as described herein may be used in embodiments of the method.

In some embodiments, the means for strengthening is adapted to be placed in a femur at a typical fracture region comprising at least one of the subcapital region of the femur, the cervical region of the femur, the basal region of the femur, and the pertrochaneric region of the femur. Some embodiments of the method comprise placing the means for strengthening in a femur at a typical fracture region, wherein the typical fracture region comprises at least one of the subcapital region of the femur, the cervical region of the femur, the basal region of the femur, and the pertrochaneric region of the femur.

In some embodiments of methods provided herein, the method further comprises forming a cavity in the bone. In some embodiments of methods provided herein, the method further comprises affixing a fastening element to a distal end of a cavity formed in the bone. Fastening elements as described herein may be used in the methods provided herein. Some embodiments of the method provided herein may be performed minimally invasively.

Provided herein is a kit for internally reinforcing a bone. In some embodiments, the kit comprises a set of implantable elements having a distal end and a proximal end for reinforcing a bone. Some embodiments of the kit comprises at least one device described herein. The kit may comprise a first implantable pad for securing the distal end of the rod within the bone. The kit may comprise a second implantable pad for securing the proximal end of the rod within the bone. The implantable element of the kit may comprise at least one of a solid rod, a collapsible rod, and a coil.

The kit may comprise a means for implanting at least one implantable element in the bone. The kits may include at least one tool for implantation of the device, devices, and/or implantable elements described herein. The means for implanting the set of rods in the bone may comprise at least one of a cutting tool and a bone drill, wherein the cutting tool and bone tool are adapted to prepare the bone for implantation of at least one of an implantable element, the first implantable pad, and the second implantable pad. In some embodiments, the means for implanting is capable of creating a cavity in bone. In some embodiments, the means for implanting the set of rods in the bone is capable of creating a cavity in the bone. In some embodiments, the bone is the femur, and wherein the means for implanting the set of rods in the bone is capable of creating a cavity in the bone about along the femur head axis running through the femur neck. In some embodiments, the kit comprises a guidewire. In some embodiments, the guidewire is flexible and/or rigid. In some embodiments, the kit comprises a system and/or a device for maintaining the existing vascular and neurological structure within the bone.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a depiction of young trabecular bone and pores.

FIG. 2 shows a cross-section of normal femur bone.

FIG. 3 shows strain on a femur head under normal loading.

FIG. 4 shows a depiction of osteoporotic trabecular bone and pores.

FIG. 5A shows forces on a femur and femur head from a fall landing on the hip (the greater trochanter).

FIG. 5B shows initial forces on a greater trochanter from a fall landing on the greater trochanter.

FIG. 5C shows torsion forces on a femur from a fall landing on the greater trochanter.

FIG. 5D shows a depiction of the greater trochanter acting as a fulcrum from a fall landing on the greater trochanter.

FIG. 5E shows an anterior view of ligaments pulling on a femur from a fall landing on the greater trochanter.

FIG. 5F shows a posterior view of ligaments pulling on a femur from a fall landing on the greater trochanter.

FIG. 5G shows a depiction of tissue over the greater trochanter.

FIG. 6A shows a pad capable of absorbing, redistributing (diffusing), and/or deflecting force on a femur head from fall forces on a hip of an embodiment

FIG. 6B shows loads on a femur during a fall landing on the hip and shows a pad capable of absorbing, redistributing (diffusing), and/or deflecting the force on a femur from fall forces on the hip.

FIG. 7 depicts a pad attached to internal and/or external compression coil and/or coil system capable of absorbing an exterior impact force on a bone, deflecting an exterior impact force on a bone, diffusing an exterior impact force on a bone, strengthening a bone, and/or reinforcing a bone of an embodiment.

FIG. 8 depicts a pad attached to an internally implanted collapsible tube capable of absorbing an exterior impact force on a bone, deflecting an exterior impact force on a bone, diffusing an exterior impact force on a bone, strengthening a bone, and/or reinforcing a bone of an embodiment.

FIG. 9 depicts strengthening and/or reinforcing bone and/or areas prone to fracture of an embodiment.

FIG. 10 depicts an embodiment comprising a reinforcing element in a cavity of a femur.

FIG. 11 depicts a bar scaffold of an embodiment implanted along lines of physiological compression and/or tension forces in normal bone, and/or along lines of strain and/or stress of diseased, osteoporotic, and/or aged bone.

FIG. 12 depicts a set of bars of an embodiment implanted along lines of physiological compression and/or tension forces in normal bone, and/or along lines of strain and/or stress of diseased, osteoporotic, and/or aged bone.

FIG. 13A depicts a set of bars of an embodiment implanted along lines of physiological compression and/or tension forces in normal bone, and/or along lines of strain and/or stress of diseased, osteoporotic, and/or aged bone.

FIG. 13B shows loads on a femur head during a fall landing on the femur having a set of bars of an embodiment capable of reinforcing, and/or strengthening the bone and/or of absorbing, redistributing (diffusing), and/or deflecting the force on a femur head from fall forces on the hip.

FIG. 14A depicts a bar scaffold of an embodiment implanted in a bone.

FIG. 14B shows loads on a femur having a bar scaffold of an embodiment during a fall landing on the femur.

FIG. 15A shows an embodiment depicting reinforcing material in the femur head of a femur.

FIG. 15B depicts an embodiment showing injection of injected material into the trabecular area of a femur head.

FIG. 16 depicts a device of an embodiment including rods, a scaffold, and reinforcing material implanted in a femur, and a pad implanted substantially outside the femur.

FIG. 17 depicts an embodiment of a means for absorbing force comprising wire-type rods (wires), a scaffold, and reinforcing material implanted in a femur, and a pad implanted substantially outside the femur

FIG. 18 depicts an embodiment of a means for absorbing force comprising rods and reinforcing material implanted in a femur, and a pad implanted substantially outside the femur.

FIG. 19A-19E depicts steps of a method of implanting an embodiment.

FIG. 20 depicts an implanted pad including a rod implanted along a load line and/or implanted generally along the bone axis, and also includes a scaffold which is reinforced with reinforcing material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

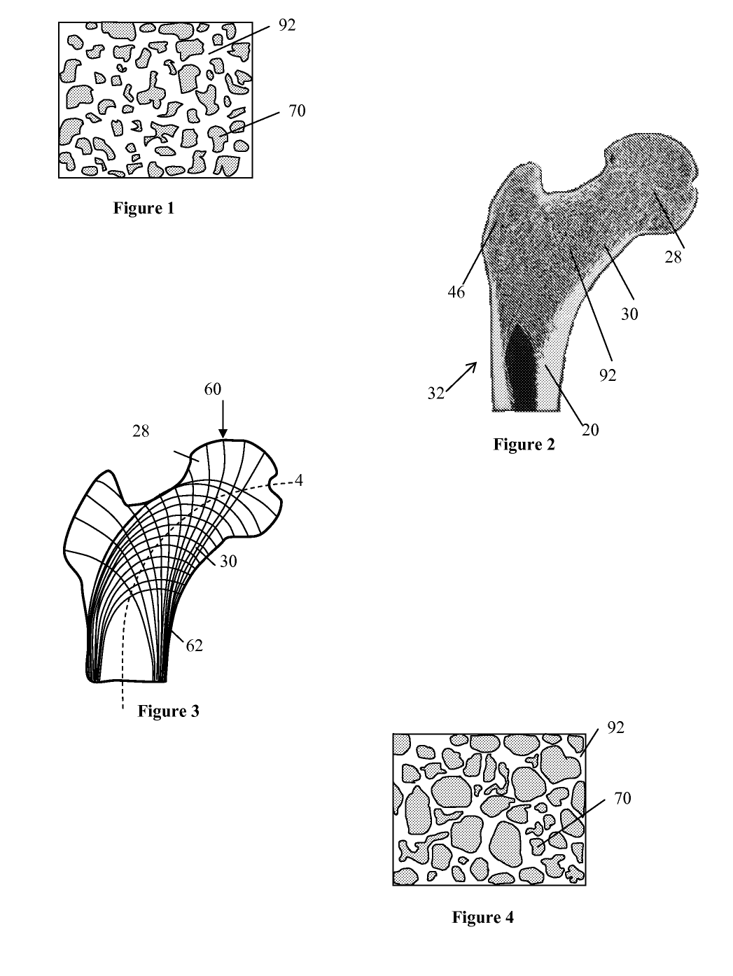

Normal bone consists of spongy bone, which can be described as open networks of branching trabecular bone which allows bone to withstand stresses in different directions. FIG. 1 shows a depiction of young trabecular bone 92 and pores 70. FIG. 2 shows a cross-section of normal femur bone 32, including the femur head 28, femoral neck 30, greater trochanter 46, and showing cortical bone 20 and trabecular (or spongy) bone 92. Trabecular bone 92 also allows the bone to be lightweight while having compression strength and tensile strength. FIG. 3 shows strain on a femur head 28 under normal loading (which may include walking, lifting, bending, sitting, running, leaning, and standing). Also shown in FIG. 3 are load lines 62 that are typical based on normal loading, for example, such as load (shown by arrow 60) which is directed primarily on the femoral head 28, and translates through the femoral neck 30. Spongy bone strength is aided by collagen fibers combined with mineral deposits. The collagen fibers are weaker relative to the mineral deposits but are also more flexible, whereas the mineral deposits are stronger than the collagen bur are also more brittle than collagen. In osteoporotic (and/or aging bone) bone, there is a decrease in bone mineral density, with an decrease in trabecular bone, decrease in bone mineral deposits resulting in a decrease in the bone structure to diffuse force and an increase in stiffness. FIG. 4 shows a depiction of osteoporotic trabecular bone 92 and pores 70. A comparison of FIG. 1 to FIG. 4 shows larger pores and/or less trabecular bone generally in the osteoporotic bone.

Certain people are more likely to develop osteoporosis than others. Osteoporosis may be caused by various hormonal conditions, smoking and medications (specifically glucocorticoids) as well as many chronic diseases. Factors that increase the likelihood of developing osteoporosis and fractures include: personal history of fracture after age 50, current low bone mass, history of fracture in a first degree relative (for example, a biological parent), being female, being thin and/or having a small frame, advanced age, a family history of osteoporosis, estrogen deficiency as a result of menopause, especially early or surgically induced, abnormal absence of menstrual periods (amenorrhea), anorexia nervosa, low lifetime calcium intake, vitamin D deficiency, use of certain medications (corticosteroids, chemotherapy, anticonvulsants and others), presence of certain chronic medical conditions, low testosterone levels in men, an inactive lifestyle, current cigarette smoking, excessive use of alcohol, and being Caucasian or Asian, although African Americans and Hispanic Americans are at significant risk as well.

Additionally, there are changes in bone structure that occur in osteoporotic or aging bone. These changes result in reduced ability to withstand forces of a fall by diffusing the force of the fall within the spongy bone. The decrease in bone mineral density in osteoporotic and/or aging bone results in a loss of or a decreased ability to deflect the forces of a fall, of which 85% of the force is typically directed to the hip. Femoral neck and greater trochanter fractures occur because of the forces on a bone resulting from a fall on the greater trochanter.

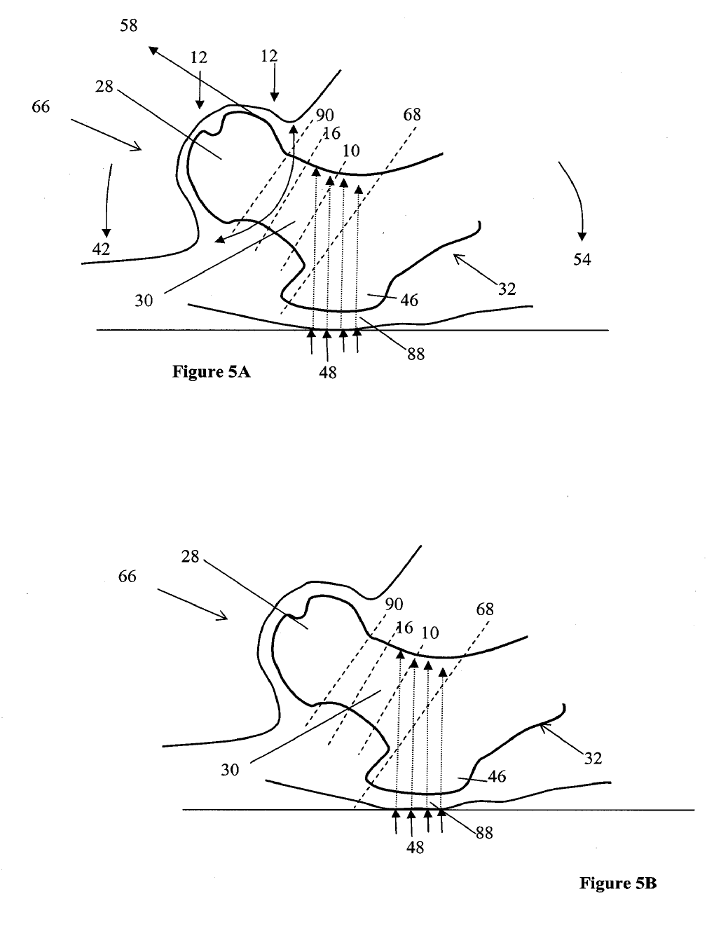

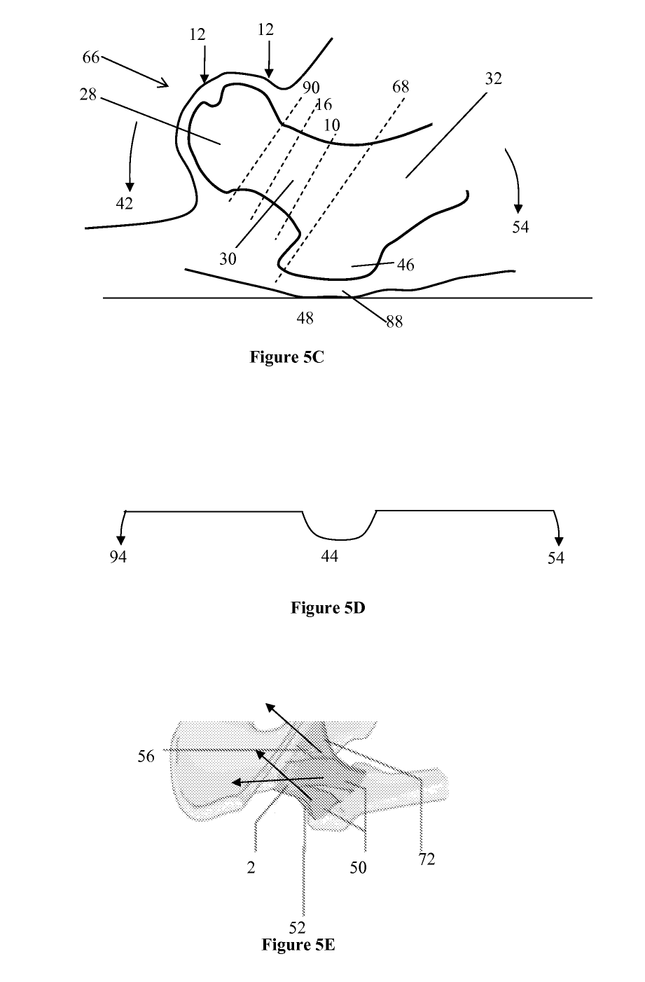

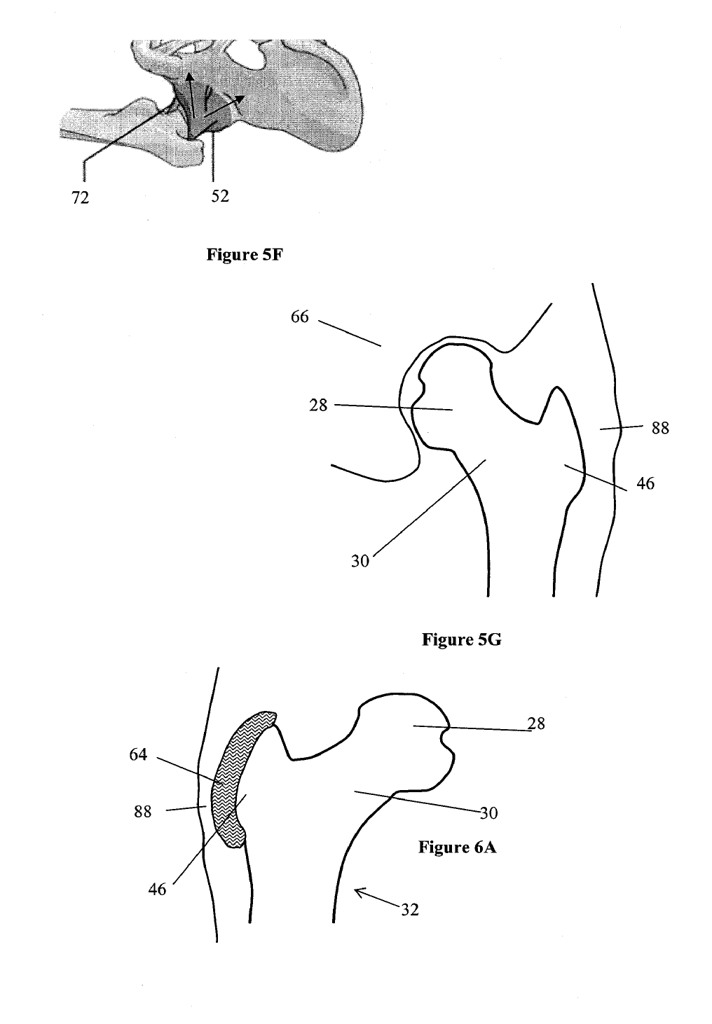

FIG. 5A shows forces (shown by arrows 12, 66, 54), on a femur 32 and femur head 28 from a fall landing on the hip (the greater trochanter 46). There is a combination of forces on the femur when a person fall's on their hip. The combination of forces creates a strong concentration of pull and/or shear on the femoral neck 30 around the greater trochanter 46. As a person falls on their greater trochanter 46, the ground exerts a force 48 which translates through the soft tissue 80 at the greater trochanter 46, and then through the femur 32. As the person fully lands, their legs put force on the femur toward the ground 54, and the torso, pelvis 66, and upper body put additional forces on the femur toward the ground 12. This can create a fracture, most typically in the femoral neck 30, which usually breaks in one of four locations, called the subcapital fracture site 90, cervical fracture site 16, basal fracture site 10, and the pertrochanteric fracture site 68. FIG. 5B shows initial forces on a greater trochanter 46 from a fall landing on the greater trochanter 46. As depicted in FIG. 5B, initially, as the hip impacts the ground 48, typically at the greater trochanter 48, the impact creates upward force (shown by arrows in the figure) which is focused on a small area and travels upward through the femur 32. FIG. 5C shows torsional forces 42, 54 on a femur 32 from a fall landing on the greater trochanter 46. As depicted, when the greater trochanter 46 area of the body hits the ground 48, the weight of pelvis 66 and upper body on one end and leg on other end create torsional forces around greater trochanter 46. The greater trochanter becomes fulcrum. This can create a fracture, most typically in the femoral neck 30, which usually breaks in one of four locations, called the subcapital fracture site 90, cervical fracture site 16, basal fracture site 10, and the pertrochanteric fracture site 68. FIG. 5D shows a depiction of the greater trochanter acting as a fulcrum 44 from a fall landing on the greater trochanter when the upper body and the leg both exert downward (and other) forces 94, 54, while the greater trochanter is fixed against the ground. There are many ligaments which add to this equation, creating various forces on the femur and the pelvis during a fall. FIG. 5E shows an anterior view of ligaments, including the ligament of the head of the femur 56, the acetabular labrum 2, the ischiofemoral ligament 52, the iliofemoral ligament 50, and the pubofemoral ligament 72, pulling (shown by arrows) on a femur from a fall landing on the greater trochanter. FIG. 5F shows a posterior view of ligaments, including the pubofemroal ligament 72 and the ischiofemroal ligament 52 pulling (shown by arrows) on a femur from a fall landing on the greater trochanter. The weight and pull of the leg distally and the weight and pull of the upper body create tension in ligaments and result in pulling on the femoral head and neck in various directions (depicted, for non-limiting example, by arrows in FIG. 5E and FIG. 5F).

Another factor leading to bone fracture in osteoporotic patients (particularly fractures in the femoral neck and greater trochanter fractures in a fall on the hip, although other fractures to other bones are contemplated herein) is the change in bone causing weakness and inability to diffuse force to other stronger parts of the bone and of the body. As patients age the structure, density, and make-up of their trabecular bone and cortical bone change, the elastic buckling capacities their bones also reduce. The outer diameter of aging osteoporotic bone expands and cortical thinning occurs and, thus, the bending strength of the patient's bone can be conserved only up to a point-a point less than the bending strength of a bone of a non-osteoporotic patient. As a result, buckling, which typically occurs prior to a fracture, occurs sooner in an osteoporotic bone than in healthy non-osteoporotic bone, and a decrease in bone strength occurs (between a 10% and a 60% decrease). Furthermore, the bone experiences increased stiffness (less elasticity) as the bone mass decreases and there is a decrease in bone mineral density. As a result, osteoporotic bones are more at risk of fracture, particularly when a patient experiences a fall.

In addition to reduced ability to diffuse force, elderly tend to fall directly on hips (typically, the greater trochanter). Elderly are likely to be relatively thin and have less skin/soft tissue padding over greater trochanter than younger people/adults. Regardless of age, there is not much skin/soft tissue 88 padding over the greater trochanter 46, shown in FIG. 5G. The scarcity of skin/soft padding means that when a person falls and lands on the greater trochanter, the forces of the fall are directed to the bone, and not diffused, absorbed, or deflected to other areas of the body which may be able to withstand the fall forces.

Thus, not only are elderly more likely to have osteoporosis, but they are more likely to fall on their hips, they have less padding to absorb the fall external to the bone, and their internal bone structure is less able to withstand, absorb, diffuse, and/or deflect the forces of the fall.

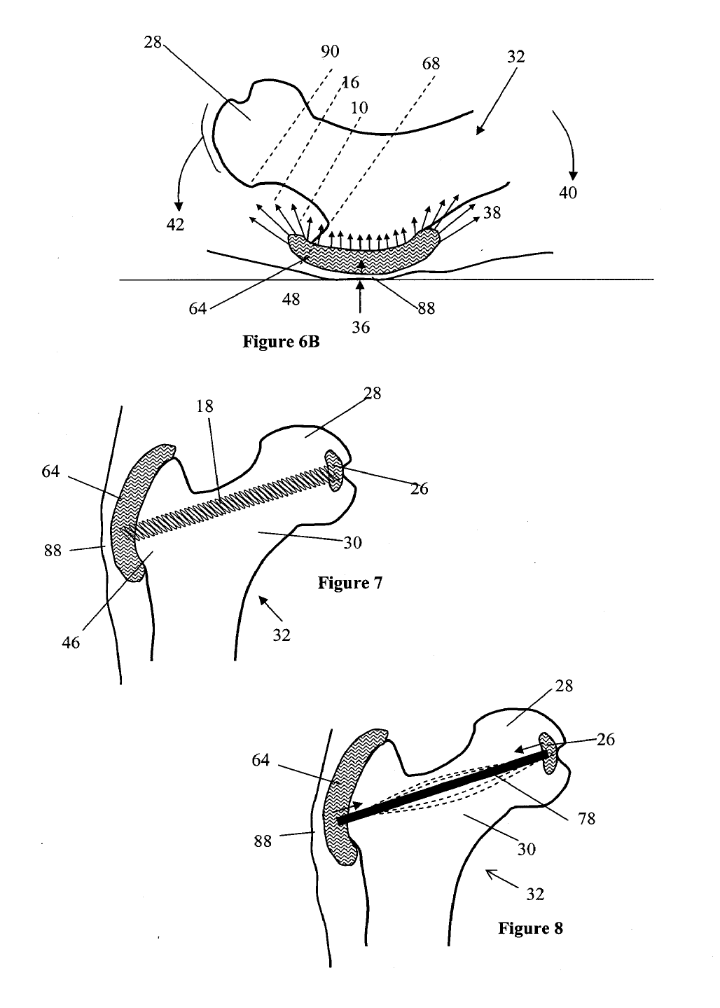

Provided herein is a device comprising an implantable pad. In some embodiments, the pad is adapted to absorb an exterior impact force on a bone, deflect an exterior impact force on a bone, diffuse an exterior impact force on a bone, strengthen a bone, and/or reinforce a bone. FIG. 6A shows a pad 64 capable of absorbing, redistributing (diffusing), and/or deflecting force on a femur 32, including the femur head 28 and femur neck 30, from fall forces on a hip of an embodiment. A pad may be implanted outside of the bone (such as between bone and connective tissue or on top of connective tissue). For non-limiting example, the pad 64 may be implanted at areas of greater trochanter 46 where impact with the ground occurs. A pad device 64 may be a liquid or a gel which hardens. It may be a pre-formed pad. It may be a bladder-type device which may be implanted minimally invasively, and filled once the bladder is implanted with a liquid or gel or hardening material or curing material. The pad 64 provides distribution of force over a larger area, and thus decreases, distributes, absorbs, and/or diffuses the forces on the greater trochanter and on the femur. FIG. 6B shows loads (forces—depicted by arrows in FIG. 6B) on a femur 32 during a fall landing on the hip and shows a pad 64 capable of absorbing, redistributing (diffusing), and/or deflecting the force on a femur from fall forces on the hip. Forces shown (but are not limited to), include forces from the upper body and pelvis 42, forces from legs and lower body 40, and the forces from hitting the ground 38 which may be absorbed, diffused, and/or deflected by a pad 64. Typical fractures which may be avoided by this include breaks that typically occur in one of four locations, i.e. the subcapital fracture site 90, cervical fracture site 16, basal fracture site 10, and the pertrochanteric fracture site 68.

Provided herein is a device comprising a coil having a portion adapted to be implanted in a bone. In some embodiments, the device further comprises an implantable pad adapted to interface with the bone. In some embodiments, the pad and the coil are adapted to cooperate in absorbing an exterior impact force on a bone, deflecting an exterior impact force on a bone, diffusing an exterior impact force on a bone, strengthening a bone, and/or reinforcing a bone. In some embodiments, the coil has limited axial elasticity, whereby the rod may be stretched or compressed axially, but compressed only to the point where the coil is completely compressed (coil loops touching) when straight but compressed. In some embodiments the coil may be bent laterally. In some embodiments, the coil may be wound in a direction that lengthens the coil and reduces the coil loop diameter prior to implantation. In some embodiments, the coil loop diameter expands from a pre-implantation diameter to an implanted diameter. In some embodiments, the coil engages a cavity wall of the bone upon expansion to its implanted diameter. In some embodiments, the coil may be implanted into a pre-made cavity of a bone.

The coil may be adapted to substantially align with at least one direction of load in the bone experienced in a bone during a fall. The coil may be adapted to substantially align with at least one direction of load in the bone experienced in normal use. Normal use may include, for non-limiting example, at least one of walking, lifting, bending, sitting, running, leaning, and standing.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is the pad. In some embodiments the closure element interfaces with the pad and the coil.

FIG. 7 depicts a pad 64 attached to internal and/or external compression coil 18 and/or coil system capable of absorbing an exterior impact force on a bone, deflecting an exterior impact force on a bone, diffusing an exterior impact force on a bone, strengthening a bone, and/or reinforcing a bone of an embodiment. The embodiment depicted in FIG. 7 has a fastening element 26 attached within the femoral head 28 to the bone of the femur 32, and a coil 18 attached to the fastening element 26. The coil 18 is implanted through the femoral neck 30, and exits the greater trochanter 46 into the pad 64, which is implanted substantially outside the greater trochanter 46 and at least a portion of which is adjacent to soft tissue 88 and seals the bone where the coil 18 has been inserted. The pad 64 acts as a closure element. The coil 18 and/or the pad 64 can compress upon impact to the greater trochanter 46, thus diffusing, deflecting, and absorbing the forces of impact (including a fall) on the greater trochanter. This can protect the femoral neck and the femur generally from breaking at typical fracture sites 90, 16, 10, 68 (shown, for example, in FIG. 6B).

Provided herein is a device comprising a rod having a portion adapted to be implanted in a bone. In some embodiments, the device further comprises an implantable pad adapted to interface with the bone. The pad and the rod may be adapted to cooperate in absorbing, deflecting, and/or diffusing force on the bone. In some embodiments, the rod is collapsible along its axis. In some embodiments, the rod has axial elasticity. In some embodiments, the axial elasticity is created by a coil. In some embodiments the axial elasticity is due to material properties of the rod. In some embodiments, the rod is made of a superelastic material, such as a memory metal. In some embodiments, the rod has axial rigidity. In some embodiments, the rod has lateral flexibility. In some embodiments, the rod has lateral elasticity.

The rod may be adapted to substantially align with at least one direction of load in the bone experienced in a bone during a fall. In some embodiments, the rod is adapted to substantially align with at least one direction of load in the bone experienced in normal use. Normal use may include, for non-limiting example, at least one of walking, lifting, bending, sitting, running, leaning, and standing.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is the pad. In some embodiments the closure element interfaces with the pad and the rod.

FIG. 8 depicts a pad 64 attached to an internally implanted collapsible tube 78 capable of absorbing an exterior impact force on a bone such as a femur 32, deflecting an exterior impact force on a bone such as a femur 32, diffusing an exterior impact force on a bone such as a femur 32, strengthening a bone such as a femur 32, and/or reinforcing a bone such as a femur 32 of an embodiment. In the embodiment shown in FIG. 8, the rod 78 can compress axially and can flex, or bend laterally within the femoral neck (shown as dotted lines in the figure). The rod 78 of an embodiment as shown in FIG. 8 may be hollow (a tube), and/or collapsible. The embodiment depicted in FIG. 8 has a fastening element 26 attached within the femoral head 28 to the bone, and a rod 78 (collapsible or flexible) attached to the fastening element 26. The rod 78 is implanted through the femoral neck 30, and exits the greater trochanter into the pad 64, which is implanted substantially outside the greater trochanter and at least a portion of which is adjacent to soft tissue 88 and seals the bone where the rod 78 has been inserted. The pad 64 acts as a closure element. The pad 64 can compress upon impact to the greater trochanter, thus diffusing, deflecting, and absorbing the forces of impact (including a fall) on the greater trochanter). The rod 64 can compress and/or deflect within the bone upon impact to the greater trochanter, thus diffusing, deflecting, and absorbing the forces of impact (including a fall) on the greater trochanter. This can protect the femoral neck and the femur generally from breaking at typical fracture sites 90, 16, 10, 68 (shown, for example, in FIG. 6B).

Provided herein is a device comprising a multiple-rod scaffold adapted to be implanted within a bone. The scaffold may comprise a plurality of rods. The scaffold may comprise a plurality of wires. The scaffold may comprise a plurality of coils. The scaffold may comprise any combination of rods, wires, and/or coils. In some embodiments, at least one of the rods, wires, and coils of the scaffold is adapted to be implanted substantially parallel relative to another rod, wire, or coil of the scaffold. At least one of the rods, wires, and/or coils is adapted to be implanted at a displaced position relative to another rod, wire, and/or coil of the scaffold.

At least one of the rods, coils, and/or wires may be adjustable in length.

In some embodiments, the scaffold is adapted to substantially align with at least one direction of load in the bone experienced in a bone during a fall. The scaffold may be adapted to substantially align with at least one direction of load in the bone experienced in normal use. Normal use may include, for non-limiting example, at least one of walking, lifting, bending, sitting, running, leaning, and standing. The scaffold may be capable of absorbing an exterior impact force on a bone, deflecting an exterior impact force on a bone, diffusing an exterior impact force on a bone, strengthening a bone, and/or reinforcing a bone.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is an implantable pad. In some embodiments the closure element interfaces with the pad and the scaffold.

In some embodiments, the device further comprises an implantable pad adapted to interface with the bone, as described herein.

FIG. 11 depicts a bar scaffold 82 of an embodiment implanted generally along lines 62 of physiological compression and/or tension forces in normal bone (for example, a femur 32), and/or along lines of strain and/or stress of diseased, osteoporotic, and/or aged bone. In this embodiment, multiple flexible wires 82 have been inserted into the femur at about the lines of tensile force 62 experienced in normal use. The scaffold 82 of the embodiment shown in FIG. 11 not only provides absorption, deflection, and/or diffusion of forces of a fall, but it additionally and/or alternatively reinforces and/or strengthens the bone. The device of FIG. 11 may further comprise at least one of a reinforcing material, a means for strengthening the bone, and a means for reinforcing the bone, as described herein. The device of FIG. 11 may be capable of reinforcing and/or strengthening bone to withstand forces of normal use (as described herein), and forces of a fall.

In some embodiments, the scaffold comprises flexible wires which simulate flexible collagen fibers, and bend or compress with tension and/or compression. The scaffold can comprise compression spring coils (coils) which also can flex and compress to simulate or replace collagen fibers in the bone. The location that the scaffold can be placed includes, but is not limited to, along the tensile force lines of normal use, or the tensile and compressive force lines of a fall. The scaffold is capable of absorbing, deflecting, and diffusing forces within a bone from normal use and/or forces experienced during a fall. The scaffold may be attached to the bone by a fastening element, such as an adherent or a pad, as described herein.

The scaffold may be implanted in areas prone to fracture. If implanted in a femur, for non-limiting example, this may include the femoral neck, the greater trochanter. The scaffold may be comprised of materials and porosity which mimics trabecular bone/spongy bone. Thus, a scaffold may comprise a network of wires or bone cement with pores allowing the network to absorb forces and flex in compression. The network may comprise flexible wires or fibers, such as wires or fibers made of memory metals. The scaffold may be able to manage (deflect, diffuse, absorb) forces from multiple directions. The scaffold structure may allow for placement along lines of stress, but also provide cross-linking and bracing of areas of the bone prone to fracture, (such as areas shown in the femur in FIG. 6B). The scaffold may be placed to reinforce areas prone to fracture. The scaffold may be attached to the bone by a fastening element, such as an adherent or a pad attached to the wall.

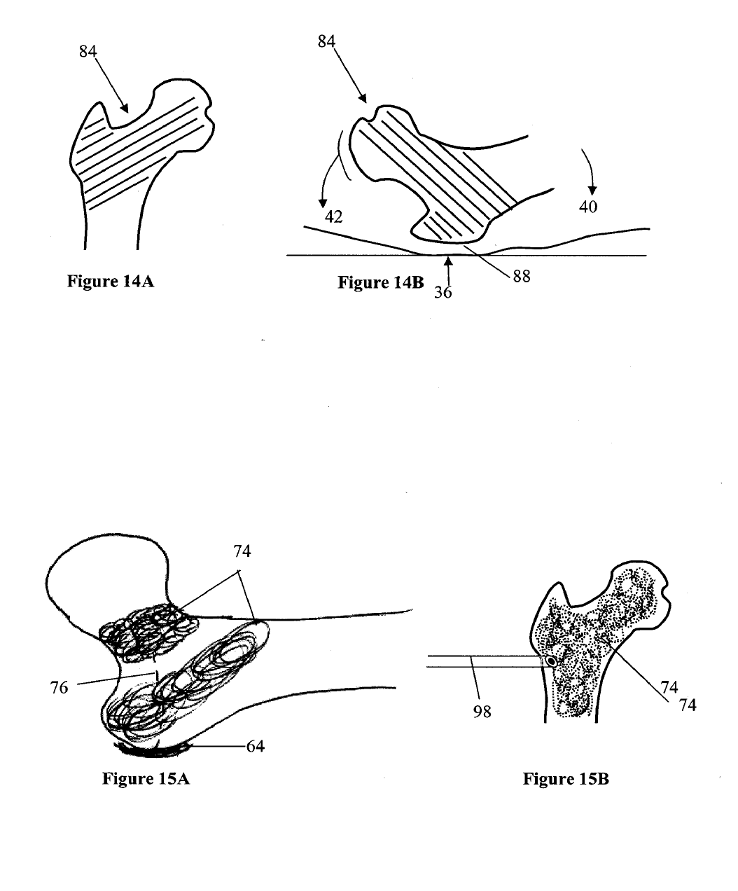

FIG. 14A depicts a bar scaffold 84 of an embodiment implanted in a bone (a femur). In the embodiment shown in FIG. 14A, a plurality of rods, wires, or coils has been placed in the femur to reinforce the bone from impacts such as a fall or normal use forces. FIG. 14B shows loads 36, 42, 40 on a femur having a bar scaffold 84 of an embodiment during a fall landing on the femur (landing on the soft tissue 88 near the greater trochanter). The scaffold 84 of the embodiments shown in FIG. 14A and FIG. 14B not only provide absorption, deflection, and/or diffusion of forces of a fall, but also reinforce and/or strengthen the bone by replacing and/or adding to trabecular bone which has deteriorated over time or due to disease or other factors noted herein. The device of FIG. 14 may be capable of reinforcing and/or strengthening bone to withstand forces of normal use (as described herein), and forces of a fall.

Provided herein is a device comprising an implant adapted to be implanted in a bone at a first orientation and a first position relative to the bone.

The device may comprise a deployable element adapted to deploy in the bone at a second orientation and second position relative to the bone. The deployable element may be coupled to the implant. The deployable element may unfold, or fold. The deployable element may be adapted to deploy in the bone. The deployable element may expand radially from the implant, and/or expand distally from the implant. The deployable element may be adapted to deploy at least a portion of the deployable element outside the bone.

The deployable element may deploy within the femur head. The deployable element may be a support for the bone under compression and/or under strain. The deployable element may provide support for the bone under compression and/or under strain. The deployable element may increase the yield strength of the bone at about where the deployable element is deployed.

FIG. 12 depicts a set of bars 8a, 8b of an embodiment implanted along lines 62 of physiological compression and/or tension forces in normal bone, and/or along lines 62 of strain and/or stress of diseased, osteoporotic, and/or aged bone. In this example the set of bars 8a, 8b are implanted in the femur through the femur head 28 and at least partially through and/or into the femoral neck 30 of the femur. The embodiment of FIG. 12 may be implanted in a cavity (not shown) in the bone from the greater trochanter approximately along the lines of stress and/or strain as shown. The bar within the femoral head may be deployable from the bar extending from about the greater trochanter to the femoral head. The bars of the embodiment show provide reinforcement of the bone along normal compression and/or tension force lines from normal use. The bars may also provide reinforcement of the bone to withstand forces from a fall including, but not limited to, the impact forces on the hip and greater trochanter, the forces from ligaments, and the forces from the upper and lower body weight of the person who fell. The device of FIG. 12 may be capable of reinforcing and/or strengthening bone to withstand forces of normal use (as described herein), and forces of a fall.

FIG. 13A depicts a set of bars 6, 24 of an embodiment implanted along lines of physiological compression and/or tension forces in normal bone, and/or along lines of strain and/or stress of diseased, osteoporotic, and/or aged bone. The embodiment of FIG. 13A may be implanted in a cavity (not shown) in the bone from the greater trochanter approximately along an axis shown through the femoral neck as shown. The bar within the femoral head 6 may be deployable from the bar 24 extending from about the greater trochanter to the femoral head, generally along the axis 4 of the femur. The bars of the embodiment show provide reinforcement of the bone. The bars may also provide reinforcement of the bone to withstand forces from a fall including, but not limited to, the impact forces on the hip and greater trochanter, the forces from ligaments, and the forces from the upper and lower body weight of the person who fell. FIG. 13B shows loads 42, 36, 40 on a femur head during a fall landing on the femur (on the soft tissue 88 near the greater trochanter) having a set of bars 6, 24 of an embodiment capable of reinforcing, and/or strengthening the bone and/or of absorbing, redistributing (diffusing), and/or deflecting the force on a femur head from fall forces on the hip. The devices of FIG. 13A and/or FIG. 13B may be capable of reinforcing and/or strengthening bone to withstand forces of normal use (as described herein), and forces of a fall

The deployable element may be a generally ring-shaped support once deployed within the bone. The generally ring-shaped support may generally abut cortical bone of the bone, or be implanted where spongy bone interfaces with cortical bone. The ring may be metal (including but not limited to, titanium, stainless steel, memory metals such as nitinol). The ring may be a plastic material. The ring may be placed in the internal bone in the femoral neck and/or the greater trochanter region to provide reinforcement of the wall of the bone (cortical bone). The ring may correspond in size and shape to the internal bone of the region in which it is implanted. This may be because the ring conforms when implanted, or the region may be measured prior to implantation, and the ring may be chosen, adjusted, or altered based on the measurements taken. The ring may adhere to the wall by tension, an adherent, or by penetrating the pores of the bone. A fastening element, such as those described herein may be used to adhere the ring and/or the rod to the wall of the bone.

The deployable element may be implanted into a cavity in the bone. The cavity may be pre-formed prior to implantation of the support, or be formed by the generally ring-shaped support. The cavity may exist prior to implantation of the implant. The cavity may be formed by, for non-limiting example, at least one of cutting, drilling, and compressing the bone, which may include trabecular and/or cortical bone. Other bone cavitation tools and methods of using the tools are contemplated herein. The cavity may be formed by drilling a hole in the bone, inserting a pressurizable bladder in the trabecular bone area of the bone, inflating the bladder in the hole, and compressing the trabecular bone generally toward the cortical bone by pressurizing the bladder. In another embodiment of the method, the cavity may be formed by drilling a hole in the bone, expanding the drilled hole by at least one of digging the trabecular bone out, and drilling additional areas of bone at an angle to the hole, using the hole drilled as an access point. Bone matter displaced by the cavity may and/or may not be removed from the bone.

In an embodiment wherein the bone is a femur, the deployed deployable element may be a generally ring-shaped support within at least one of the femur head, the femur neck, and/or anywhere along the axis defined by the femur head, femur neck and femur body. The deployed deployable element may be a cylinder-like support within at least one of the femur head, the femur neck, or anywhere along the axis defined by the femur head, femur neck and femur body.

In some embodiments, the deployable element is a scaffold, as describe herein. In some elements the scaffold may be reinforced by a reinforcing element. In some embodiments, the reinforcing element comprises bone cement, and/or collagen. In some embodiment the scaffold comprises a plurality of wires. In some embodiments the scaffold comprises a wire mesh.

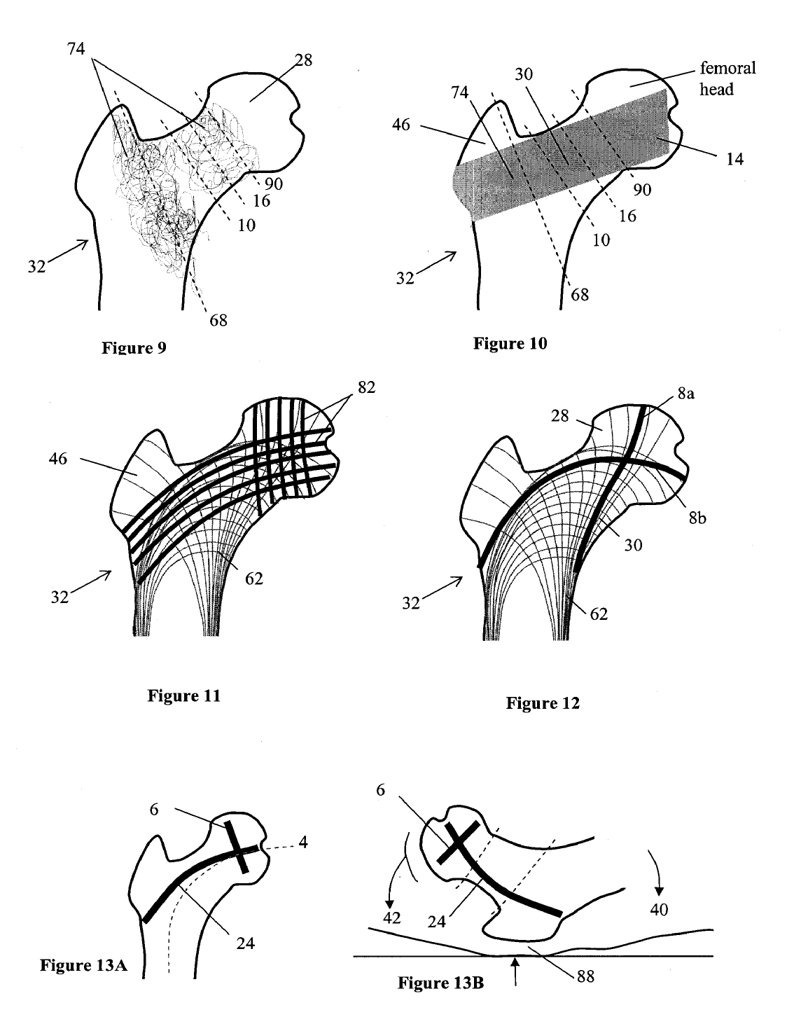

FIG. 9 depicts strengthening and/or reinforcing bone and/or areas prone to fracture of an embodiment. A bone cement, foam, elastomer, scaffold of wires or filaments, or another material which can be injected into a space and cured or harden in place may be used. In the embodiment shown, the reinforcing element (reinforcing material) 74 has been placed into the femoral neck at about the locations of typical fractures of the femur 32, including but not limited to the subcapital region 90, the cervical region 16, the basal region 10, and the pertrochaneric region 68. The device of FIG. 9 may be capable of reinforcing and/or strengthening bone to withstand forces of normal use (as described herein), and forces of a fall.

FIG. 10 depicts an embodiment comprising a reinforcing element (reinforcing material) 74 in a cavity 14 of a femur 32. The cavity 14 was created minimally invasively, and a filled with a reinforcing element (material) 74 such as a bone cement, for a non-limiting example. The reinforcing element as shown in this embodiment is placed substantially where the typical fractures occur in the head 28, neck 30, and at a portion of the greater trochanter 46 of the femur 32, including but not limited to the subcapital region 90, the cervical region 16, the basal region 10, and the pertrochaneric region 68. The device of FIG. 10 may be capable of reinforcing and/or strengthening bone to withstand forces of normal use (as described herein), and forces of a fall.

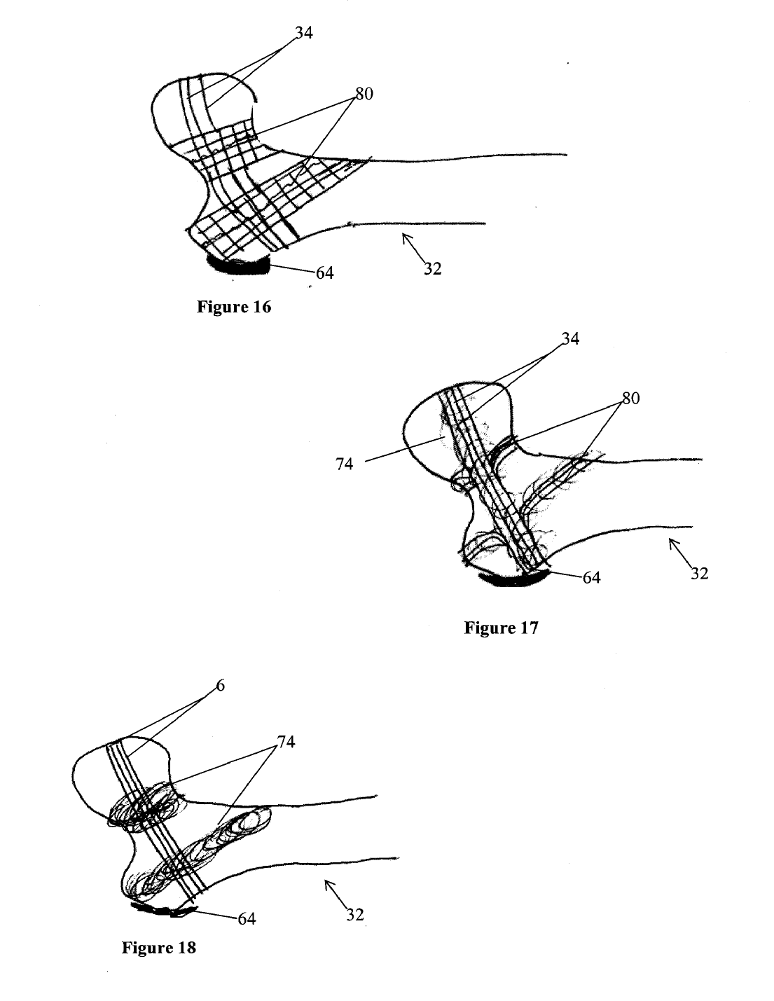

FIG. 16 depicts a device of an embodiment including rods 34, a scaffold 80, and reinforcing material implanted in a femur 32, and a pad 64 implanted substantially outside the femur. In the embodiment depicted in FIG. 16, the rods 34 have been inserted, and the scaffold 80 has been deployed from at least one rod in the areas most likely to fracture (for example, in at least one of the subcapital region, the cervical region, the basal region, and the pertrochaneric region). The scaffold may unfold, fold, and/or deploy. The scaffold may expand radially from a rod, and/or it may expand distally from a rod. The scaffold may further be reinforced by a porous material, such as a foam or a bone cement, for non-limiting example. The embodiment of FIG. 16 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 16 may be capable of reinforcing and/or strengthening bone to withstand forces of normal use (as described herein), and forces of a fall.

At least one of the implant and the deployable element of the implant may be adapted to substantially align with at least one direction of load in the bone experienced in a bone during a fall. At least one of the implant and the deployable element of the implant may be adapted to substantially align with at least one direction of load in the bone experienced in normal use. Normal use may include, for non-limiting example, at least one of walking, lifting, bending, sitting, running, leaning, and standing. In some embodiments, at least one of the implant and the deployable element is capable of absorbing an exterior impact force on a bone, deflecting an exterior impact force on a bone, diffusing an exterior impact force on a bone, strengthening a bone, and/or reinforcing a bone.

In some embodiments, the device comprises a fastening element capable of fastening a distal end of the coil to spongy bone and/or trabecular bone and/or cortical bone of the bone. The fastening element may comprise at least one of bone cement, an elastomer, a glue, and a spongy foam. The fastening element may comprise at least one of a corkscrew fixating element, a nail, and a staple. In some embodiments, the generally ring-shaped support is also the fastening element, comprised of least one of bone cement, an elastomer, a glue, and a spongy foam. In some embodiments, the generally ring-shaped support comprises a fastening element which may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is an implantable pad. In some embodiments the closure element interfaces with the pad and the implant.

In some embodiments, the device further comprises an implantable pad adapted to interface with the bone, as described herein.

Provided herein is a device comprising an means for maintaining in position an means for absorbing a force on a bone. In some embodiments the force results from a fall. In some embodiments, the force results from normal use. Normal use may include, for non-limiting example, at least one of walking, lifting, bending, sitting, running, leaning, and standing. The means for absorbing may be implantable. The means for maintaining in position may be implantable.

The means for absorbing may comprise at least one of a pad, a rod, a collapsible rod, and a coil. The means for absorbing may comprise at least one of an elastomer, a silicone, a memory metal, and spongy foam. The means for absorbing may comprise a material having a higher yield strength than the bone. The means for absorbing may comprise a material having about the same yield strength as trabecular bone. The means for absorbing may comprise a material having about the same yield strength as cortical bone. The means for absorbing may comprise at least one of metal and bone cement.

FIG. 6A shows a means for absorbing force comprising a pad capable of absorbing, redistributing (diffusing), and/or deflecting force on a femur head from fall forces on a hip of an embodiment. A pad 64 may be implanted outside of the bone (such as between bone and connective tissue or on top of connective tissue). For non-limiting example, the pad may be implanted at areas of greater trochanter where impact with the ground occurs. A pad device may be a liquid or a gel which hardens. It may be a pre-formed pad. It may be a bladder-type device which may be implanted minimally invasively, and filled once the bladder is implanted with a liquid or gel or hardening material or curing material. The pad provides distribution of force over a larger area, and thus decreases, distributes, absorbs, and/or diffuses the forces on the greater trochanter and on the femur. FIG. 6B shows loads (forces—depicted by arrows in FIG. 6B) on a femur during a fall landing on the hip and shows a pad capable of absorbing, redistributing (diffusing), and/or deflecting the force on a femur from fall forces on the hip.

FIG. 7 depicts an embodiment of a means for absorbing force comprising a coil 18 and a pad 64. The pad is attached to internal and/or external compression coil and/or coil system capable of absorbing, redistributing (diffusing), and/or deflecting force from a fall of an embodiment. The embodiment depicted in FIG. 7 has a fastening element 26 attached within the femoral head 28 to the bone, and a coil 18 attached to the fastening element 26. The coil 18 is implanted through the femoral neck 30, and exits the greater trochanter 46 into the pad 64, which is implanted substantially outside the greater trochanter 46 adjacent to soft tissue 88 and seals the bone where the coil 18 has been inserted. The pad 64 acts as a closure element. The coil and/or the pad can compress upon impact to the greater trochanter, thus diffusing, deflecting, and absorbing the forces of impact (including a fall) on the greater trochanter). This can protect the femoral neck and the femur generally from breaking at typical fracture sites (shown, for example, in FIG. 6B).

FIG. 8 depicts an embodiment of a means for absorbing force comprising a rod 78 and a pad 64. The pad is attached to an internally implanted collapsible tube (rod) capable of absorbing, redistributing (diffusing), and/or deflecting force from a fall of an embodiment. In the embodiment shown in FIG. 8, the rod 78 can compress axially and can flex, or bend laterally within the femoral neck. The rod 78 of an embodiment as shown in FIG. 8 may be hollow (a tube), and/or collapsible. The embodiment depicted in FIG. 8 has a fastening element 26 attached within the femoral head 28 to the bone, and a rod 78 (collapsible or flexible) attached to the fastening element 26. The rod is implanted through the femoral neck, and exits the greater trochanter into the pad 64, which is implanted substantially outside the greater trochanter adjacent to soft tissue 88 and seals the bone where the rod has been inserted. The pad acts as a closure element. The pad can compress upon impact to the greater trochanter, thus diffusing, deflecting, and absorbing the forces of impact (including a fall) on the greater trochanter). The rod can compress and/or deflect within the bone upon impact to the greater trochanter, thus diffusing, deflecting, and absorbing the forces of impact (including a fall) on the greater trochanter. This can protect the femoral neck and the femur generally from breaking at typical fracture sites (shown, for example, in FIG. 6B).

FIG. 16 depicts an embodiment of a means for absorbing force comprising rods 34, a scaffold 80, and reinforcing material implanted in a femur 32, and a pad 64 implanted substantially outside the femur. In the embodiment of FIG. 16, the rods 34 have been inserted, and the scaffold 80 has been deployed from at least one rod in the areas most likely to fracture (for example, in at least one of the subcapital region, the cervical region, the basal region, and the pertrochaneric region). The scaffold may unfold, fold, and/or deploy. The scaffold may expand radially from a rod, and/or it may expand distally from a rod. The scaffold may further be reinforced by a porous material, such as foam or bone cement, for non-limiting example. The embodiment of FIG. 16 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 16 may be capable of reinforcing and/or strengthening bone to withstand forces of normal use (as described herein), and forces of a fall.

FIG. 17 depicts an embodiment of a means for absorbing force comprising wire-type rods (wires) 34, a scaffold 80, and reinforcing material 74 implanted in a femur 32, and a pad 64 implanted substantially outside the femur 32. In the embodiment of FIG. 17, the wires have been inserted, and the scaffold has been deployed from at least one wire in the areas most likely to fracture (for example, in at least one of the subcapital region, the cervical region, the basal region, and the pertrochaneric region). The scaffold 80 of FIG. 17 comprises wires. The scaffold 80 may unfold, fold, and/or deploy. The scaffold 80 may expand radially from a wire-type rod 34, and/or it may expand distally from a wire-type rod 34. The scaffold 80 may further be reinforced by a porous material 74, such as foam or bone cement, for non-limiting example. The embodiment of FIG. 17 shows additionally a pad 64 substantially outside the bone at the greater trochanter 46 region of the femur 32. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall. The scaffold may comprise at least one rod. Rods may include rods and/or coils which may compress longitudinally, expand longitudinally, or deflect laterally. In some embodiments, a rod may be a coil as described herein. In some embodiments, the rod may be a solid rod. In some embodiments a rod may be a collapsible rod.

FIG. 18 depicts an embodiment of a means for absorbing force comprising rods 6 and reinforcing material 74 implanted in a femur 32, and a pad 64 implanted substantially outside the femur 32 at the greater trochanter 46. In the embodiment of FIG. 18, the rods 6 have been inserted through the femoral neck and reinforcing material has been implanted at about the locations of typical fractures, including, but not limited to at least one of the subcapital region, the cervical region, the basal region, and the pertrochaneric region of the femur. A pad 64, such as one of any embodiment described herein, has been implanted to interface with the greater trochanter 46. The device of FIG. 18 may be capable of reinforcing and/or strengthening bone to withstand forces of normal use (as described herein), and forces of a fall.

FIG. 20 depicts an implanted pad including a rod 78 implanted along a load line. The rod 78 may also or alternatively be implanted generally along the bone axis. FIG. 20 also includes a scaffold 80 which is reinforced with reinforcing material 74. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

The means for maintaining in position may comprise a fastening element capable of fastening a distal end of the means for absorbing to spongy bone and/or trabecular bone, and/or cortical bone of the bone. The fastening element may comprise at least one of bone cement, an elastomer, glue, and spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, glue, and spongy foam. The closure element may comprise at least one of a corkscrew fixating element, a nail, and a staple. In some embodiments, the closure element is the means for absorbing. In some embodiments the closure element interfaces with the means for maintaining in position and the means for absorbing.

In some embodiments, the means for maintaining in position is an implantable pad adapted to interface with the bone, as described herein. In some embodiments, the means for absorbing a force comprises an implantable pad adapted to interface with the bone, as described herein.

Provided herein is a device comprising an implantable means for maintaining in position a means for deflecting a force on a bone. In some embodiments the force results from a fall. In some embodiments, the force results from normal use. Normal use may include, for non-limiting example, at least one of walking, lifting, bending, sitting, running, leaning, and standing.

In some embodiments, the means for deflecting the force on the bone deflects the force in a direction whereby the force is directed to at least one of a portion of the bone that can withstand the force without fracturing the bone, another body part without fracturing the bone, and/or a material external to the body without fracturing the bone. The means for deflecting the force on the bone may deflect the force to at least one of a portion of the bone that is sufficiently strong to withstand the force without fracturing the bone, and a portion of a body that is sufficiently strong to withstand the force without fracturing the bone and without fracturing the portion of the body.

The portion of the bone may be reinforced with at least one of bone cement, silicone, metal, including memory metal, and elastomers. The portion of the body may be reinforced with at least one of bone cement, silicone, metal, including memory metal, and elastomers. In some embodiments, the device comprises a reinforcing element capable of reinforcing the portion of the bone, wherein the reinforcing element comprises at least one of bone cement, silicone, metal, including memory metal, and elastomers. In some embodiments, the device comprises a reinforcing element capable of reinforcing the portion of the body, wherein the reinforcing element comprises at least one of bone cement, silicone, metal, including memory metal, and elastomers.

In some embodiments, the means for maintaining in position comprises a fastening element capable of fastening a distal end of the means for deflecting to spongy bone and/or trabecular bone, and/or cortical bone of the bone. In some embodiments, the fastening element comprises at least one of bone cement, an elastomer, a glue, and a spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is the means for deflecting. In some embodiments the closure element interfaces with the means for maintaining in position and the means for deflecting.

In some embodiments, the means for maintaining in position is an implantable pad adapted to interface with the bone, as described herein. In some embodiments, the means for deflecting a force comprises an implantable pad adapted to interface with the bone, as described herein.

FIG. 6A shows a means for absorbing force comprising a pad 64 capable of absorbing, redistributing (diffusing), and/or deflecting force on a femur head 28 from fall forces on a hip of an embodiment. A pad may be implanted outside of the bone (such as between bone and connective tissue or on top of connective tissue). For non-limiting example, the pad may be implanted at areas of greater trochanter 46 where impact with the ground occurs. A pad device may be a liquid or a gel which hardens. It may be a pre-formed pad. It may be a bladder-type device which may be implanted minimally invasively, and filled once the bladder is implanted with a liquid or gel or hardening material or curing material. The pad provides distribution of force over a larger area, and thus decreases, distributes, absorbs, and/or diffuses the forces on the greater trochanter and on the femur. FIG. 6B shows loads 36, 38, 40, 42 (forces—depicted by arrows in FIG. 6B) on a femur during a fall landing on the hip and shows a pad capable of absorbing, redistributing (diffusing), and/or deflecting the force on a femur from fall forces on the hip.

FIG. 7 depicts an embodiment of a means for deflecting force comprising a coil 18 and a pad 64. The pad is attached to internal and/or external compression coil 18 and/or coil system capable of absorbing, redistributing (diffusing), and/or deflecting force from a fall of an embodiment. The embodiment depicted in FIG. 7 has a fastening element 26 attached within the femoral head 28 to the bone, and a coil 18 attached to the fastening element 26. The coil is implanted through the femoral neck 30, and exits the greater trochanter 46 into the pad 64, which is implanted substantially outside the greater trochanter 46 and seals the bone (femur 32) where the coil 18 has been inserted. The pad 64 acts as a closure element. The coil 18 and/or the pad 64 can compress upon impact to the greater trochanter 46 and the soft tissue 88 adjacent the greater trochanter 46, thus diffusing, deflecting, and absorbing the forces of impact (including a fall) on the greater trochanter). This can protect the femoral neck and the femur generally from breaking at typical fracture sites 90, 16, 10, 68 (shown, for example, in FIG. 6B).

FIG. 8 depicts an embodiment of a means for deflecting force comprising a rod 78 and a pad 64. The pad 64 is attached to an internally implanted collapsible tube (rod) 78 capable of absorbing, redistributing (diffusing), and/or deflecting force from a fall of an embodiment. In the embodiment shown in FIG. 8, the rod 78 can compress axially and can flex, or bend laterally within the femoral neck 30. The rod 78 of an embodiment as shown in FIG. 8 may be hollow (a tube), and/or collapsible. The embodiment depicted in FIG. 8 has a fastening element 26 attached within the femoral head 28 to the bone, and a rod 78 (collapsible or flexible) attached to the fastening element 26. The rod 78 is implanted through the femoral neck 30, and exits the greater trochanter into the pad 64, which is implanted substantially outside the greater trochanter and seals the bone (the femur 32 in FIG. 8) where the rod 78 has been inserted. The pad 64 acts as a closure element. The pad 64 can compress upon impact to the greater trochanter and the soft tissue 88 adjacent the greater trochanter, thus diffusing, deflecting, and absorbing the forces of impact (including a fall) on the greater trochanter). The rod 78 can compress and/or deflect within the bone upon impact to the greater trochanter and the soft tissue 88 adjacent the greater trochanter, thus diffusing, deflecting, and absorbing the forces of impact (including a fall) on the greater trochanter. This can protect the femoral neck 30 and the femur generally from breaking at typical fracture sites 90, 16, 10, 68 (shown, for example, in FIG. 6B).

FIG. 11 depicts an embodiment of a means for deflecting force comprising a bar scaffold 82 implanted along lines 62 of physiological compression and/or tension forces in normal bone, and/or along lines 62 of strain and/or stress of diseased, osteoporotic, and/or aged bone (a femur 32, for example). In this embodiment, multiple flexible wires have been inserted into the femur 32 at about the lines of tensile force 62 experienced in normal use. The scaffold 82 of the embodiment shown in FIG. 11 not only provides absorption, deflection, and/or diffusion of forces of a fall, but also and/or alternatively reinforces and/or strengthens the bone. The device of FIG. 11 may further comprise at least one of a reinforcing material, a means for strengthening the bone, and a means for reinforcing the bone, as described herein. The device of FIG. 11 may be capable of reinforcing and/or strengthening bone to withstand forces of normal use (as described herein), and forces of a fall.

FIG. 14A depicts an embodiment of a means for deflecting force comprising a scaffold of bars 84 implanted in a bone. FIG. 14A depicts a bar scaffold 84 of an embodiment implanted in a bone. In the embodiment shown in FIG. 14A, a plurality of rods, wires, or coils has been placed in the femur to reinforce the bone from impacts such as a fall or normal use forces. FIG. 14B depicts an embodiment of a means for deflecting force showing loads 36, 42, 40 on a femur having a bar scaffold 84 of an embodiment during a fall landing on the soft tissue 88 adjacent to the greater trochanter of a femur. FIG. 14B shows loads on a femur having a bar scaffold 84 of an embodiment during a fall landing on the femur. The scaffold of the embodiments shown in FIG. 14A and FIG. 14B not only provide absorption, deflection, and/or diffusion of forces of a fall, but also reinforce and/or strengthen the bone by replacing and/or adding to trabecular bone which has deteriorated over time or due to disease or other factors noted herein. The devices of FIG. 14A and FIG. 14B may be capable of reinforcing and/or strengthening bone to withstand forces of normal use (as described herein), and forces of a fall.

FIG. 16 depicts an embodiment of a means for deflecting force comprising rods or flexible wires 34, a scaffold 80, and reinforcing material implanted in a femur, and a pad 64 implanted substantially outside the femur 32. In the embodiment of FIG. 16, the rods or flexible wires 34 have been inserted, and the scaffold 80 has been deployed from at least one rod (flexible wire) in the areas most likely to fracture (for example, in at least one of the subcapital region, the cervical region, the basal region, and the pertrochaneric region). The scaffold 80 may unfold, fold, and/or deploy. The scaffold 80 may expand radially from a rod, and/or it may expand distally from a rod. The scaffold 80 may further be reinforced by a porous material, such as a foam or a bone cement, for non-limiting example. The embodiment of FIG. 16 shows additionally a pad 64 substantially outside the bone. In any embodiment described herein having such a pad 64, the pad 64 may be partially within the bone, and/or the pad 64 may be comprised by the closure element to close the access point of the bone through which any of the internally implanted elements of the device were implanted. The device of FIG. 16 may be capable of reinforcing and/or strengthening bone to withstand forces of normal use (as described herein), and forces of a fall.

FIG. 17 depicts an embodiment of a means for absorbing force comprising wire-type rods (wires) 34, a scaffold 80, and reinforcing material 74 implanted in a femur 32, and a pad 64 implanted substantially outside the femur at the greater trochanter 46. In the embodiment of FIG. 17, the wires 34 have been inserted, and the scaffold 80 has been deployed from at least one wire in the areas most likely to fracture (for example, in at least one of the subcapital region, the cervical region, the basal region, and the pertrochaneric region). The scaffold 80 of FIG. 17 comprises wires. The scaffold 80 may unfold, fold, and/or deploy. The scaffold 80 may expand radially from a wire-type rod, and/or it may expand distally from a wire-type rod. The scaffold 80 may further be reinforced by a porous material, such as a foam or a bone cement, for non-limiting example. The embodiment of FIG. 17 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall.

FIG. 18 depicts an embodiment of a means for absorbing force comprising rods 6 and reinforcing material 74 implanted in a femur 32 at the greater trochanter 46, and a pad 64 implanted substantially outside the femur 32. In the embodiment of FIG. 18, the rods 6 have been inserted through the femoral neck and reinforcing material 74 has been implanted at about the locations of typical fractures, including, but not limited to the subcapital region, the cervical region, the basal region, and the pertrochaneric region of the femur 32. A pad 64, such as one of any embodiment described herein, has been implanted to interface with the greater trochanter. The device of FIG. 18 may be capable of reinforcing and/or strengthening bone to withstand forces of normal use (as described herein), and forces of a fall.

FIG. 20 depicts an embodiment of a means for absorbing force comprising an implanted pad including a rod 78 implanted along a load line. The rod 78 may also or alternatively be implanted generally along the bone axis. The means for absorbing force shown in FIG. 20 also includes a scaffold 80 which is reinforced with reinforcing material 74. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

Provided herein is a device comprising an means for maintaining in position a means for diffusing a force on a bone. The means for maintaining in position may be implantable. The means for diffusing a force on a bone may be implantable. In some embodiments the force results from a fall. In some embodiments, the force results from normal use. Normal use may include, for non-limiting example, at least one of walking, lifting, bending, sitting, running, leaning, and standing.

In some embodiments, the means for maintaining in position comprises a fastening element capable of fastening a distal end of the means for deflecting to spongy bone and/or trabecular bone, and/or cortical bone of the bone. In some embodiments, the fastening element comprises at least one of bone cement, an elastomer, a glue, and a spongy foam. The fastening element may comprise at least one of a corkscrew fixating element, a nail, and a staple.

In some embodiments, the device comprises a closure element capable of sealing the bone at the proximal end of the implanted device. The closure element may comprise at least one of bone cement, an elastomer, a glue, and a spongy foam. The closure element may comprise at least one of a corkscrew fixating element, a nail, and a staple. In some embodiments, the closure element is the means for diffusing. In some embodiments the closure element interfaces with the means for maintaining in position and the means for diffusing.

In some embodiments the force is from a fall. In some embodiments, the force is from normal loading of the bone. Normal loading may comprise loads from, for non-limiting example, walking, lifting, bending, sitting, running, leaning, and standing.

In some embodiments, the means for maintaining in position is an implantable pad adapted to interface with the bone, as described herein. In some embodiments, the means for diffusing a force comprises an implantable pad adapted to interface with the bone, as described herein.

FIG. 6A shows a means for diffusing force comprising a pad 64 capable of absorbing, redistributing (diffusing), and/or deflecting force on a femur head 28 from fall forces on a hip of an embodiment. A pad may be implanted outside of the bone (such as between bone and connective tissue or on top of connective tissue). For non-limiting example, the pad may be implanted at areas of greater trochanter where impact with the ground occurs. A pad device may be a liquid or a gel which hardens. It may be a pre-formed pad. It may be a bladder-type device which may be implanted minimally invasively, and filled once the bladder is implanted with a liquid or gel or hardening material or curing material. The pad provides distribution of force over a larger area, and thus decreases, distributes, absorbs, and/or diffuses the forces on the greater trochanter and on the femur. FIG. 6B shows loads 36, 38, 42, 40 (forces—depicted by arrows in FIG. 6B) on a femur during a fall landing on the hip and shows a pad capable of absorbing, redistributing (diffusing), and/or deflecting the force on a femur from fall forces on the hip.

FIG. 7 depicts an embodiment of a means for diffusing force comprising a coil 18 and a pad 64. The pad is attached to internal and/or external compression coil 18 and/or coil system capable of absorbing, redistributing (diffusing), and/or deflecting force from a fall of an embodiment. The embodiment depicted in FIG. 7 has a fastening element 26 attached within the femoral head 28 to the bone, and a coil 18 attached to the fastening element 26. The coil 18 is implanted through the femoral neck 30, and exits the greater trochanter 46 into the pad 64, which is implanted substantially outside the greater trochanter 46 and seals the bone (femur 32) where the coil 18 has been inserted. The pad 64 acts as a closure element. The coil 18 and/or the pad can compress upon impact to the greater trochanter 46 and the soft tissue 88 adjacent the greater trochanter 46, thus diffusing, deflecting, and absorbing the forces of impact (including a fall) on the greater trochanter). This can protect the femoral neck and the femur generally from breaking at typical fracture sites 90, 16, 10, 68 (shown, for example, in FIG. 6B).

FIG. 8 depicts an embodiment of a means for diffusing force comprising a rod 78 and a pad 64. The pad 64 attached to an internally implanted collapsible tube (rod) capable of absorbing, redistributing (diffusing), and/or deflecting force from a fall of an embodiment. In the embodiment shown in FIG. 8, the rod 78 can compress axially and can flex, or bend laterally within the femoral neck 30. The rod 78 of an embodiment as shown in FIG. 8 may be hollow (a tube), and/or collapsible. The embodiment depicted in FIG. 8 has a fastening element 26 attached within the femoral head 28 to the bone, and a rod 78 (collapsible or flexible) attached to the fastening element 26. The rod 78 is implanted through the femoral neck 30, and exits the greater trochanter into the pad 64, which is implanted substantially outside the greater trochanter and seals the bone (the femur 32 in FIG. 8) where the rod 78 has been inserted. The pad 64 acts as a closure element. The pad 64 can compress upon impact to the greater trochanter and the soft tissue 88 adjacent the greater trochanter, thus diffusing, deflecting, and absorbing the forces of impact (including a fall) on the greater trochanter). The rod can compress and/or deflect within the bone upon impact to the greater trochanter and the soft tissue 88 adjacent the greater trochanter, thus diffusing, deflecting, and absorbing the forces of impact (including a fall) on the greater trochanter. This can protect the femoral neck 30 and the femur generally from breaking at at least one of the typical fracture sites 90, 16, 10, 68 (shown, for example, in FIG. 6B).

FIG. 11 depicts an embodiment of a means for diffusing force comprising a bar scaffold 84 implanted along lines 62 of physiological compression and/or tension forces in normal bone, and/or along lines 62 of strain and/or stress of diseased, osteoporotic, and/or aged bone (a femur 32, for example). In this embodiment, multiple flexible wires have been inserted into the femur 32 at about the lines of tensile force 62 experienced in normal use. The scaffold 82 of the embodiment shown in FIG. 11 not only provides absorption, deflection, and/or diffusion of forces of a fall, but also and/or alternatively reinforces and/or strengthens the bone. The device of FIG. 11 may further comprise at least one of a reinforcing material, a means for strengthening the bone, and a means for reinforcing the bone, as described herein. The device of FIG. 11 may be capable of reinforcing and/or strengthening bone to withstand forces of normal use (as described herein), and forces of a fall.

FIG. 14A depicts an embodiment of a means for diffusing force comprising a scaffold of bars 84 implanted in a bone. FIG. 14A depicts a bar scaffold 84 of an embodiment implanted in a bone. In the embodiment shown in FIG. 14A, a plurality of rods, wires, or coils has been placed in the femur to reinforce the bone from impacts such as a fall or normal use forces. FIG. 14B depicts an embodiment of a means for diffusing force showing loads 36, 40, 42 on a femur 32 having a bar scaffold 84 of an embodiment during a fall landing on the soft tissue 88 adjacent to the greater trochanter of a femur. FIG. 14B shows loads on a femur having a bar scaffold 84 of an embodiment during a fall landing on the femur. The scaffold of the embodiments shown in FIG. 14A and FIG. 14B not only provide absorption, deflection, and/or diffusion of forces of a fall, but also reinforce and/or strengthen the bone by replacing and/or adding to trabecular bone which has deteriorated over time or due to disease or other factors noted herein. The device of FIG. 14A and FIG. 14B may be capable of reinforcing and/or strengthening bone to withstand forces of normal use (as described herein), and forces of a fall.

FIG. 16 depicts an embodiment of a means for diffusing force comprising rods or flexible wires 34, a scaffold 80, and reinforcing material implanted in a femur, and a pad 64 implanted substantially outside the femur 32. In the embodiment of FIG. 16, the rods or flexible wires 34 have been inserted, and the scaffold 80 has been deployed from at least one rod (flexible wire) in the areas most likely to fracture (for example, in at least one of the subcapital region, the cervical region, the basal region, and the pertrochaneric region). The scaffold 80 may unfold, fold, and/or deploy. The scaffold 80 may expand radially from a rod, and/or it may expand distally from a rod. The scaffold 80 may further be reinforced by a porous material, such as a foam or a bone cement, for non-limiting example. The embodiment of FIG. 16 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 16 may be capable of reinforcing and/or strengthening bone to withstand forces of normal use (as described herein), and forces of a fall.

FIG. 17 depicts an embodiment of a means for diffusing force comprising wire-type rods (wires) 34, a scaffold 80, and reinforcing material 74 implanted in a femur 32, and a pad 64 implanted substantially outside the femur 32 at the greater trochanter 46. In the embodiment of FIG. 17, the wires 34 have been inserted, and the scaffold 80 has been deployed from at least one wire in the areas most likely to fracture (for example, in at least one of the subcapital region, the cervical region, the basal region, and the pertrochaneric region). The scaffold 80 of FIG. 17 comprises wires. The device of the embodiment of FIG. 17 may imitate collagen fiber, including having flexibility and bending ability, allowing some deflection of forces. The scaffold may unfold, fold, and/or deploy. The scaffold may expand radially from a wire-type rod, and/or it may expand distally from a wire-type rod. The scaffold may further be reinforced by a porous material, such as a foam or a bone cement, for non-limiting example. The embodiment of FIG. 17 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall.

FIG. 18 depicts an embodiment of a means for diffusing force comprising rods 6 and reinforcing material 74 implanted in a femur 32, and a pad 64 implanted substantially outside the femur 32 at the greater trochanter 46. In the embodiment of FIG. 18, the rods 6 have been inserted through the femoral neck and reinforcing material 74 has been implanted at about the locations of typical fractures, including, but not limited to the subcapital region, the cervical region, the basal region, and the pertrochaneric region of the femur 32. A pad 64, such as one of any embodiment described herein, has been implanted to interface with the greater trochanter. The device of FIG. 18 may be capable of reinforcing and/or strengthening bone to withstand forces of normal use (as described herein), and forces of a fall.

FIG. 20 depicts an embodiment of a means for diffusing force comprising an implanted pad including a rod 78 implanted along a load line. The rod 78 may also or alternatively be implanted generally along the bone axis. The means for diffusing force in FIG. 20 also includes a scaffold 80 which is reinforced with reinforcing material 74. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

In any embodiment provided or contemplated herein, the bone may be a bone of the hip and/or a pelvic bone. The bone may be a leg bone, including, for non-limiting example, a femur, a tibia, a fibula. The bone may be a bone of the arm or wrist, including, for non-limiting example, a radius, an ulna, a carpal, a metacarpal, and/or a humerus. The bone may be a bone of the spine, including, for non-limiting example, a vertebra. The bone may be a bone of the feet. The bone may any one of the scapula, clavicle, and/or a rib.

Provided herein is a device comprising an implantable means for maintaining in position a means for reinforcing a bone, wherein the means for reinforcing is capable of reinforcing the bone to withstand a force on the bone. In some embodiments, the means for reinforcing is adapted to be implanted in a location in the bone of high stress and/or a location in the bone of high strain during a fall. In some embodiments, the means for reinforcing is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for reinforcing is adapted to be implanted in a location in the bone of high stress and/or a location in the bone of high strain during a fall. In some embodiments, the means for reinforcing is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for reinforcing comprises at least one of a pad, a rod, a collapsible rod, and a coil. In some embodiments, the means for reinforcing comprises at least one of an elastomer, a silicone, a memory metal, and spongy foam. The means for reinforcing may comprise a material having a higher yield strength than bone. The means for reinforcing may comprise a material having about the same yield strength as trabecular bone. In some embodiments, the means for reinforcing comprises a material having about the same yield strength as cortical bone. In some embodiments, the means for reinforcing comprises at least one of metal and bone cement.

In some embodiments, the means for maintaining in position comprises a fastening element capable of fastening a distal end of the means for reinforcing to spongy bone and/or trabecular bone, and/or cortical bone of the bone. The fastening element may comprise at least one of bone cement, an elastomer, a glue, and a spongy foam. The fastening element may comprise at least one of a corkscrew fixating element, a nail, and a staple.

Provided herein is a device comprising an implantable means for maintaining in position a means for strengthening a bone, wherein the means for strengthening is capable of strengthening the bone to withstand a force on the bone. In some embodiments, the means for strengthening is adapted to be implanted in a location in the bone of high stress and/or a location in the bone of high strain during a fall. In some embodiments, the means for strengthening is adapted to be implanted in a location in the bone of high stress and/or a location in the bone of high strain during a normal use. Normal use may include, for non-limiting example, at least one of walking, lifting, bending, sitting, running, leaning, and standing.

In some embodiments, the means for strengthening comprises at least one of a pad, a rod, a collapsible rod, and a coil. In some embodiments, the means for strengthening comprises at least one of an elastomer, a silicone, a memory metal, and spongy foam. The means for strengthening may comprise a material having a higher yield strength than bone. The means for strengthening may comprise a material having about the same yield strength as trabecular bone. In some embodiments, the means for strengthening comprises a material having about the same yield strength as cortical bone. In some embodiments, the means for strengthening comprises at least one of metal and bone cement.

In some embodiments, the means for maintaining in position comprises a fastening element capable of fastening a distal end of the means for strengthening to spongy bone and/or trabecular bone, and/or cortical bone of the bone. The fastening element may comprise at least one of bone cement, an elastomer, a glue, and a spongy foam. The fastening element may comprise at least one of a corkscrew fixating element, a nail, and a staple.

FIG. 16 depicts an embodiment of a means for reinforcing and/or strengthening a bone comprising rods or flexible wires 34, a scaffold 80, and material implanted in a femur, and a pad 64 implanted substantially outside the femur 32. In the embodiment of FIG. 16, the rods have been inserted, and the scaffold 80 has been deployed from at least one rod in the areas most likely to fracture (for example, in at least one of the subcapital region, the cervical region, the basal region, and the pertrochaneric region). The scaffold may unfold, fold, and/or deploy. The scaffold may expand radially from a rod, and/or it may expand distally from a rod. The scaffold may further be reinforced by a porous material, such as a foam or a bone cement, for non-limiting example. The embodiment of FIG. 16 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 16 may be capable of reinforcing and/or strengthening bone to withstand forces of normal use (as described herein), and forces of a fall.

FIG. 17 depicts an embodiment of a means for reinforcing and/or strengthening a bone (the femur, in this embodiment) comprising wire-type rods (wires) 34, a scaffold 80, reinforcing material 74 implanted in a femur 32, and a pad 64 implanted substantially outside the femur 32. In the embodiment of FIG. 17, the wires 34 have been inserted, and the scaffold 80 has been deployed from at least one wire in the areas most likely to fracture (for example, in at least one of the subcapital region, the cervical region, the basal region, and the pertrochaneric region). The scaffold of FIG. 17 comprises wires 80. The device of the embodiment of FIG. 17 may imitate collagen fiber, including having flexibility and bending ability, allowing some deflection of forces. The scaffold 80 may unfold, fold, and/or deploy. The scaffold 80 may expand radially from a wire-type rod, and/or it may expand distally from a wire-type rod. The scaffold 80 may further be reinforced by a porous material, such as a foam or a bone cement, for non-limiting example. The embodiment of FIG. 17 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall.

FIG. 18 depicts an embodiment of a means for reinforcing and/or strengthening a bone comprising rods 6 implanted in a femur 32, an injected material 74 at about the regions where typical fracture might occur, and a pad 64 implanted substantially outside the femur. In the embodiment of FIG. 18, the rods 6 have been inserted through the femoral neck and reinforcing and/or strengthening material (injected material) has been implanted at about the locations of typical fractures, including, but not limited to the subcapital region, the cervical region, the basal region, and the pertrochaneric region of the femur. A pad, such as one of any embodiment described herein, has been implanted to interface with the greater trochanter. The device of FIG. 18 may be capable of reinforcing and/or strengthening bone to withstand forces of normal use (as described herein), and forces of a fall.

FIG. 20 depicts an embodiment of a means for reinforcing and/or strengthening a bone comprising an implanted pad including a rod 78 implanted along a load line. The rod 78 may also or alternatively be implanted generally along the bone axis. The means for reinforcing and/or strengthening a bone in FIG. 20 also includes a scaffold 80 which is reinforced with reinforcing material 74. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

In some embodiments, the device absorbs the force on a second bone. In some embodiments, the device diffuses the force on a second bone. In some embodiments, the device deflects the force on a second bone. The second bone may be a bone of the hip, including, for non-limiting example, a femur or a pelvis. The second bone may be a leg bone, including, for non-limiting example, a femur, a tibia, a fibula. The second bone may be a bone of the arm or wrist, including, for non-limiting example, a radius, an ulna, a carpal, a metacarpal, and/or a humerus. The second bone may be a bone of the spine, including, for non-limiting example, a vertebra.

In some embodiments described herein comprising an implantable pad, at least a portion of the pad is adapted to be implanted inside of the bone. In some embodiments, the pad is implantable within a spongy bone portion of the bone. In some embodiments, the pad is adapted to be attached to a compact bone portion of the bone. In some embodiments, at least a portion of the pad is adapted to be implanted outside of the bone. In some embodiments, the pad is adapted to be attached to the bone. In some embodiments the pad is adapted to slide along a surface of an exterior portion of the bone.

Some embodiments of the device comprise osteogenic materials including osteogenic growth factors and/or growth factors. These materials may be factors found naturally in the body in bone or cartilage growth or maintenance or found associated with other cellular growth or maintenance, not found within the body. These materials may be used to seal/repair hole or bore or used to attach device structure internally or externally or used to strengthen bone or used to deflect and/or absorb force. Examples may include, but are not limited to, FGF, angiogenic growth factors, LDL, minerals, osteoblasts causing osteogenesis, or factors used by osteoblasts, osteoprogenitor cells, or cells or factors used to restructure bone including osteoclasts, forms of remodeling or osteolysis. The device may also be adapted to place bone marrow or bone marrow-like cells in femur to enhance blood production.

In some embodiments of the devices provided herein, the device comprises a reinforcing element. In some embodiments, the reinforcing element is bone cement. The cement may be adapted to be implanted in a location in the bone of high stress and/or a location in the bone of high strain during a fall. The cement may be adapted to be implanted in a location in the bone of high stress and/or a location in the bone of high strain during a normal use. Normal use may include, for non-limiting example, at least one of walking, lifting, bending, sitting, running, leaning, and standing. In some embodiments, the reinforcing element is a plurality of bars. In some embodiments, the reinforcing element is a plurality of flexible bars. In some embodiments, the reinforcing element is a plurality of flexible wires. In some embodiments, the reinforcing element is a plurality of superelastic wires. In some embodiments, the reinforcing element is a scaffold. In some embodiments, the reinforcing element is a plurality of coils.

FIG. 15A shows an embodiment depicting reinforcing material 74 in the femur head of a femur. The reinforcing material 74 (reinforcing element) in the embodiment depicted in FIG. 15A is placed in regions where typical fractures occur, including but not limited to, at least one of the subcapital region, the cervical region, the basal region, and the pertrochaneric region. The material may be implanted through a hole in the bone, for example, along an injection path 76. In the embodiment depicted, the hole is through the area of the greater trochanter. The implantation means (which may be an injection catheter, and/or a needle, for non-limiting example) accesses the typical fracture sites for the bone, and the reinforcing material may be injected, implanted. The material may be of the type that cures or hardens once implanted. The material may be an elastomer, mesh, porous bone cement, or another structure providing both strength and flexibility. In the embodiment shown, a pad 64 is placed at the access point to act as a closure element and to act as a pad to absorb, diffuse, and/or deflect forces on the greater trochanter. The device of FIG. 15A may be capable of reinforcing and/or strengthening bone to withstand forces of normal use (as described herein), and forces of a fall.

In some embodiments the reinforcing material may be injected into an area of the bone in which a scaffold has been implanted. The scaffold may be bounded or unbounded, such as, for example a by a bladder. The reinforcing material may be placed within the bounded bladder. The reinforcing material may partially or completely surround and/or encompass the scaffold. The reinforcing material may be more dense or strong (like Calcium deposits of trabecular bone) than the scaffold material (the scaffold may be more flexible and/or compliant (like collagen of bone). The scaffold area reinforced by the reinforcing material may strengthen the bone. Together, the scaffold and the reinforcing material may act within the bone as reinforced concrete acts in building and reinforcing a building.

FIG. 15B depicts an embodiment showing injection of injected (or reinforcing) material 74 into the trabecular area of a femur head using an injection needle 98, for example. The reinforcing material 74 may be placed in the bone without particular regard for the locations of fracture, and may be implanted in addition to the devices provided herein. The device of FIG. 15B may be capable of reinforcing and/or strengthening bone to withstand forces of normal use (as described herein), and forces of a fall.

FIG. 17 depicts an embodiment of a means for absorbing force comprising wire-type rods (wires) 34, a scaffold 80, and reinforcing material 74 implanted in a femur 32, and a pad 64 implanted substantially outside the femur 32 at the greater trochanter 46. In the embodiment of FIG. 17, the wires 34 have been inserted, and the scaffold 80 has been deployed from at least one wire in the areas most likely to fracture (for example, in at least one of the subcapital region, the cervical region, the basal region, and the pertrochaneric region). The scaffold 80 of FIG. 17 comprises wires. The device of the embodiment of FIG. 17 may imitate collagen fiber, including having flexibility and bending ability, allowing some deflection of forces. The scaffold may unfold, fold, and/or deploy. The scaffold may expand radially from a wire-type rod, and/or it may expand distally from a wire-type rod. The scaffold may further be reinforced by a porous material, such as a foam or a bone cement, for non-limiting example. The embodiment of FIG. 17 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall.

FIG. 18 depicts an embodiment of a means for absorbing force comprising rods 6 and reinforcing material 74 implanted in a femur 32, and a pad 64 implanted substantially outside the femur 32 at the greater trochanter 46. In the embodiment of FIG. 18, the rods 6 have been inserted through the femoral neck and reinforcing material 74 has been implanted at about the locations of typical fractures, including, but not limited to at least one of the subcapital region, the cervical region, the basal region, and the pertrochaneric region of the femur. A pad 64, such as one of any embodiment described herein, has been implanted to interface with the greater trochanter. The device of FIG. 18 may be capable of reinforcing and/or strengthening bone to withstand forces of normal use (as described herein), and forces of a fall.

FIG. 20 depicts an embodiment of a means for absorbing force comprising an implanted pad including a rod 78 implanted along a load line. The rod 78 may also or alternatively be implanted generally along the bone axis. The means for absorbing force shown in FIG. 20 also includes a scaffold 80 which is reinforced with reinforcing material 74. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

Provided herein is a method for reducing the risk of fracturing a bone of a patient comprising implanting a pad adapted to interface with a bone. Provided herein is a method for reducing the risk of fracturing a bone of a patient comprising implanting a device comprising a pad adapted to interface with a bone. The pad may be any pad as described herein. The device may be any device comprising a pad as described herein. The pad may be adapted to absorb an exterior impact force on a bone, deflect an exterior impact force on a bone, diffuse an exterior impact force on a bone, strengthen a bone, and/or reinforce a bone.

Provided herein is a method for reducing the risk of fracturing a bone of a patient comprising delivering a portion of a coil within a bone. Provided herein is a method for reducing the risk of fracturing a bone of a patient comprising delivering a portion of a device comprising a coil within a bone. Any coil as described herein may be used in the embodiments of the method. Any device comprising a coil as described herein may be used in the embodiments of the method. The method may further comprise implanting a pad adapted to interface with the bone. The pad may be any pad as described herein. The pad may be adapted to absorb, deflect, and/or diffuse an exterior impact force on a bone.

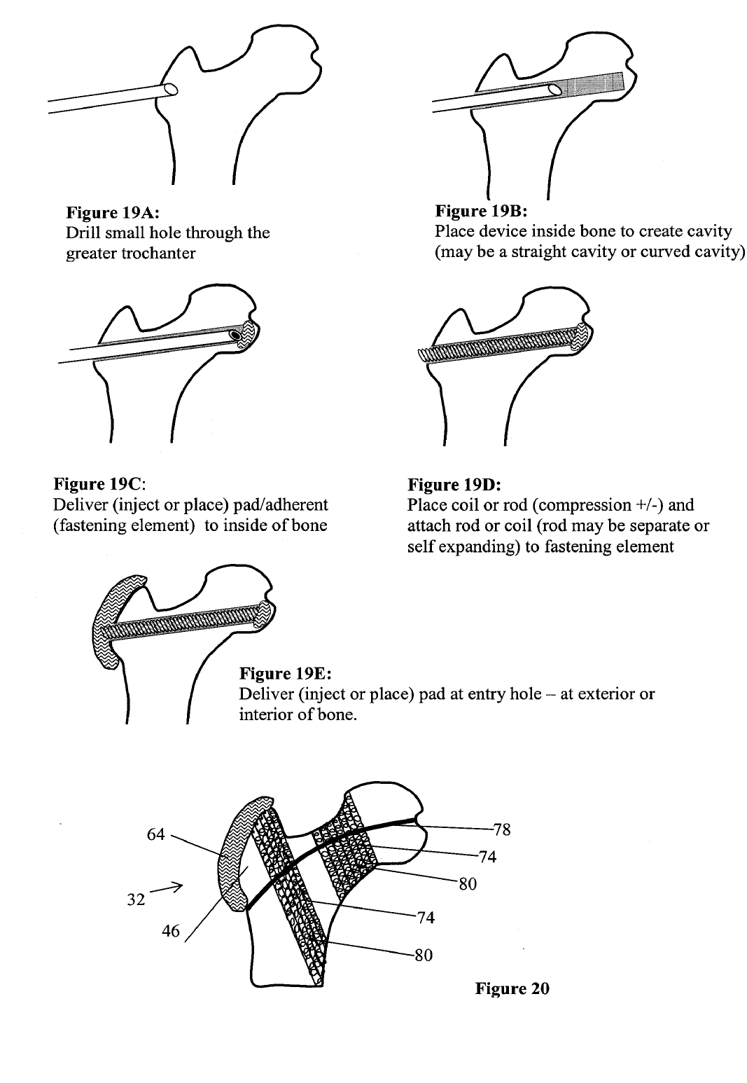

FIG. 19A-19E depicts steps of a method of implanting an embodiment. FIG. 19A depicts the step of the embodiment of drilling a small hole through the greater trochanter. FIG. 19B depicts the step of the embodiment of placing a device inside bone to create cavity (may be a straight cavity or curved cavity). FIG. 19C depicts the step of the embodiment of delivering (by injection or placing) a fastening element (such as a pad or an adherent) into the bone cavity at the distal end of the cavity against the bone (in this case, the cortical bone of the femur head). FIG. 19D depicts the step of the embodiment of placing a coil and/or rod either of which may be in compression or in tension before and/or after placement into the cavity. The step further comprises attaching the coil and/or rod to the fastening element at the distal end of the cavity. FIG. 19E depicts the step of the embodiment of delivering (by injecting or placing) a pad at the entry hole. The coil and/or rod may project through the hole into the pad. The pad may be at or in the hole.

Provided herein is a method for reducing the risk of fracturing a bone of a patient comprising delivering a portion of a rod within a bone. Provided herein is a method for reducing the risk of fracturing a bone of a patient comprising delivering a portion of a device comprising a rod within a bone. Rods and devices comprising a rod described herein may be used in the methods provided herein. The method may further comprise implanting a pad adapted to interface with the bone. The pad may be any pad as described herein. The pad may be adapted to absorb, deflect, and/or diffuse an exterior impact force on a bone. The pad and the rod may be adapted to cooperate in absorbing an exterior impact force on a bone, deflecting an exterior impact force on a bone, diffusing an exterior impact force on a bone, strengthening a bone, and/or reinforcing a bone.

Provided herein is a method for reducing the risk of fracturing a bone of a patient comprising implanting a multiple-rod scaffold within a bone. Provided herein is a method for reducing the risk of fracturing a bone of a patient comprising implanting a device comprising a multiple-rod scaffold within a bone. Multiple-rod scaffolds and devices comprising a multiple-rod scaffold as described herein may be used in the methods provided herein. The implanting may comprise orienting each rod substantially parallel to each other rod, and positioning each rod at a displaced position in the bone relative to each other rod.

Provided herein is a method for reducing the risk of fracturing a bone of a patient comprising implanting a device in a bone at a first orientation and a first position relative to the bone, wherein the device comprises an implant and the implant comprises a deployable element adapted to deploy within the bone at a second orientation and second position relative to the bone. Provided herein is a method for reducing the risk of fracturing a bone of a patient comprising implanting an implant in a bone at a first orientation and a first position relative to the bone, wherein the implant comprises a deployable element adapted to deploy within the bone at a second orientation and second position relative to the bone. Devices comprising implants having deployable elements and implants having deployable elements as described herein may be used in embodiments of the method. The method may further comprise deploying the deployable element at the second orientation and second position relative to the bone.

FIG. 17 depicts a device of an embodiment including rods (wire-type) 34, a scaffold 80, and reinforcing material 74 implanted in a femur 32, and a pad 64 implanted substantially outside the femur 32 at the greater trochanter 46. In the embodiment of FIG. 17, the wires 34 have been inserted, and the scaffold 80 has been deployed from at least one wire in the area or areas most likely to fracture (for example, in at least one of the subcapital region, the cervical region, the basal region, and the pertrochaneric region). The scaffold 80 of FIG. 17 comprises wires. The device of the embodiment of FIG. 17 may imitate collagen fiber, including having flexibility and bending ability, allowing some deflection of forces. The scaffold may unfold, fold, and/or deploy. The scaffold may expand radially from a wire-type rod, and/or it may expand distally from a wire-type rod. The scaffold may further be reinforced by a porous material, such as a foam or a bone cement, for non-limiting example. The embodiment of FIG. 17 shows additionally a pad 64 substantially outside the bone. In any embodiment described herein having such a pad, the pad may be partially within the bone, and/or the pad may be comprised by the closure element to close the access point of the bone through which any of the internally implanted elements of the device were implanted. The device of FIG. 17 may be capable of reinforcing and/or strengthening bone to withstand forces of normal use (as described herein), and forces of a fall.

Such a device as depicted in FIG. 17 may be implanted by a method provided herein. For non-limiting example, the device may be implanted by creating a hole in the greater trochanter, and inserting wires (bundled and substantially parallel at the time of implantation) into the hole. The method may comprise affixing the wires to at least one of the wall of the hole and the distal end of the hole. A fastening element may be used to affix the wires within the cavity. The wires may be unbundled to affix to the cavity walls, as the wires may be biased to a shape that is larger than the shape it has in its bundled form. The method may comprise inserting a scaffold along at least one wire. The method may comprise deploying the scaffold at set areas corresponding to at least one of the subcapital region, the cervical region, the basal region, and the pertrochaneric region, which are typical fracture sites of the femur.

An embodiment method may further comprise injecting a reinforcing material as described herein into the hole or into the area where the scaffold has been deployed. The reinforcing material may harden in place. The reinforcing material may be implanted in typical fracture sites of the bone, for example, for the case of the femur, in at least one of the subcapital region, the cervical region, the basal region, and the pertrochaneric region. The method may comprise placing a pad at the greater trochanter to close the hole, and/or to attach the wires to the pad.

Provided herein is a method for reducing the risk of fracturing a bone of a patient comprising implanting a means for absorbing force on a bone. Provided herein is a method for reducing the risk of fracturing a bone of a patient comprising implanting a device comprising a means for absorbing force on a bone. In some embodiments of the methods, the force results from a fall. In some embodiments of the methods, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for absorbing force on a bone as described herein, and means for absorbing force on a bone as described herein may be used with embodiments of the method. The method may further comprise securing the means for absorbing force within tissue. Means for securing the means for absorbing force as described herein may be used in embodiments of the method. Devices comprising means for securing the means for absorbing force as described herein may be used in embodiments of the method.

FIG. 15A shows an embodiment depicting reinforcing material 74 in the femur head of a femur. The reinforcing material 74 (reinforcing element) in the embodiment depicted in FIG. 15A is placed at typical fracture sites, including but not limited to, at least one of the subcapital region, the cervical region, the basal region, and the pertrochaneric region. The material may be implanted through a hole in the bone, for example, along an injection path 76. In the embodiment depicted, the hole is through the area of the greater trochanter. The implantation means (which may be an injection catheter, and/or a needle, for non-limiting example) accesses the typical fracture sites for the bone, and the reinforcing material may be injected, implanted. The material may be of the type that cures or hardens once implanted. The material may be an elastomer, mesh, porous bone cement, or another structure providing both strength and flexibility. In the embodiment shown, a pad 64 is placed at the access point to act as a closure element and to act as a pad to absorb, diffuse, and/or deflect forces on the greater trochanter. The device of FIG. 15A may be capable of reinforcing and/or strengthening bone to withstand forces of normal use (as described herein), and forces of a fall.

Such a device as shown in FIG. 15A may be implanted by a method provided herein. For non-limiting example, the reinforcing material may be implanted by creating a hole in the greater trochanter, and directing the injecting of a reinforcing material as described herein into the typical fracture regions of the bone, for example, for the case of the femur, in at least one of the subcapital region, the cervical region, the basal region, and the pertrochaneric region. The method may comprise placing a pad at the greater trochanter to close the hole, and/or to absorb, deflect, and/or diffuse the forces of a fall on a femur.

The method of injection of reinforcing material as described herein may be combined with any device provided herein to reinforce the bone, and/or may be combined with any method provided herein.

In some embodiments the reinforcing material may be injected into an area of the bone in which a scaffold has been implanted. The scaffold may be bounded or unbounded, such as, for example a by a bladder. The reinforcing material may be placed within the bounded bladder. The reinforcing material may partially or completely surround and/or encompass the scaffold. The reinforcing material may be more dense or strong (like Calcium deposits of trabecular bone) than the scaffold material (the scaffold may be more flexible and/or compliant (like collagen of bone). The scaffold area reinforced by the reinforcing material may strengthen the bone. Together, the scaffold and the reinforcing material may act within the bone as reinforced concrete acts in building and reinforcing a building.

FIG. 15B depicts an embodiment showing injection of injected (or reinforcing) material 74 into the trabecular area of a femur head using an injection needle 98, for example. The reinforcing material 74 may be placed in the bone without particular regard for the locations of fracture, and may be implanted in addition to the devices provided herein. For non-limiting example, the reinforcing material may be implanted by creating a hole in the greater trochanter, implanting a device through the hole, such as a device provided herein, and/or using a method provided herein, and injecting a reinforcing material as described herein through the hole.

Provided herein is a method for reducing the risk of fracturing a bone of a patient comprising implanting a means for deflecting force on a bone. Provided herein is a method for reducing the risk of fracturing a bone of a patient comprising implanting a device comprising a means for deflecting force on a bone. In some embodiments of the methods, the force results from a fall. In some embodiments of the methods, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for deflecting force on a bone as described herein, and means for deflecting force on a bone as described herein may be used with embodiments of the method. The method may further comprise securing the means for deflecting force within tissue. Means for securing the means for deflecting force as described herein may be used in embodiments of the method. Devices comprising means for securing the means for deflecting force as described herein may be used in embodiments of the method.

Provided herein is a method for reducing the risk of fracturing a bone of a patient comprising implanting a means for diffusing force on a bone. Provided herein is a method for reducing the risk of fracturing a bone of a patient comprising implanting a device comprising a means for diffusing force on a bone. In some embodiments of the methods, the force results from a fall. In some embodiments, the force results from normal use. Normal use may include, for non-limiting example, at least one of walking, lifting, bending, sitting, running, leaning, and standing. In some embodiments, the force results from normal loading of the bone. Normal loading may include loads resulting from, for non-limiting example, at least one of walking, lifting, bending, sitting, running, leaning, and standing. Devices comprising means for diffusing force on a bone as described herein, and means for diffusing force on a bone as described herein may be used with embodiments of the method. The method may further comprise securing the means for diffusing force within tissue. Means for securing the means for diffusing force as described herein may be used in embodiments of the method. Devices comprising means for securing the means for diffusing force as described herein may be used in embodiments of the method.

Provided herein is a method for reducing the risk of fracturing a bone of a patient comprising implanting a means for reinforcing a bone, and securing the means for reinforcing within tissue. In some embodiments, the means for reinforcing is capable of reinforcing the bone to withstand a force. In some embodiments, the bone is osteoporotic. In some embodiments, the force is from normal loading of the bone. Normal loading may comprise, for non-limiting example, at least one of walking, lifting, bending, sitting, running, leaning, and standing. In some embodiments, the force results from a fall. Devices comprising means for reinforcing a bone as described herein may be used with embodiments of the method. Devices comprising means for securing the means for reinforcing force as described herein may be used in embodiments of the method.

In some embodiments, the means for reinforcing is adapted to be placed in a femur at a typical fracture region comprising at least one of the subcapital region of the femur, the cervical region of the femur, the basal region of the femur, and the pertrochaneric region of the femur. Some embodiments of the method comprise placing the means for reinforcing in a femur at a typical fracture region, wherein the typical fracture region comprises at least one of the subcapital region of the femur, the cervical region of the femur, the basal region of the femur, and the pertrochaneric region of the femur.

Provided herein is a method for reducing the risk of fracturing a bone of a patient comprising implanting a means for strengthening a bone, and securing the means for strengthening within tissue. In some embodiments, the means for strengthening is capable of strengthening the bone to withstand a force. In some embodiments, the bone is osteoporotic. In some embodiments, the force is from normal loading of the bone. Normal loading may comprise, for non-limiting example, at least one of walking, lifting, bending, sitting, running, leaning, and standing. In some embodiments the force is from a fall. In some embodiments, the force results from a fall. Devices comprising means for strengthening a bone as described herein may be used with embodiments of the method. Devices comprising means for securing the means for strengthening force as described herein may be used in embodiments of the method.

In some embodiments, the means for strengthening is adapted to be placed in a femur at a typical fracture region comprising at least one of the subcapital region of the femur, the cervical region of the femur, the basal region of the femur, and the pertrochaneric region of the femur. Some embodiments of the method comprise placing the means for strengthening in a femur at a typical fracture region, wherein the typical fracture region comprises at least one of the subcapital region of the femur, the cervical region of the femur, the basal region of the femur, and the pertrochaneric region of the femur.

In some embodiments of methods provided herein, the method further comprises forming a cavity in the bone. The cavity may be formed by, for non-limiting example, at least one of cutting, drilling, and compressing the bone, which may include trabecular and/or cortical bone. Other bone cavitation tools and methods of using the tools are contemplated herein. The cavity may be formed by drilling a hole in the bone, inserting a pressurizable bladder in the trabecular bone area of the bone, inflating the bladder in the hole, and compressing the trabecular bone generally toward the cortical bone by pressurizing the bladder. In another embodiment of the method, the cavity may be formed by drilling a hole in the bone, expanding the drilled hole by at least one of digging the trabecular bone out, and drilling additional areas of bone at an angle to the hole, using the hole drilled as an access point. Bone matter displaced by the cavity may and/or may not be removed from the bone. In some embodiments of methods provided herein, the method further comprises affixing a fastening element to a distal end of a cavity formed in the bone. Fastening elements as described herein may be used in the methods provided herein. Some embodiments of the method provided herein may be performed minimally invasively.

Provided herein is a method for reducing risk of bone fracture provided herein comprising determining the force distributions of use and/or loading on a bone, and reinforcing the bone using the methods and devices provided herein to provide additional strength to the bone to reduce fracture.

Some embodiments of the methods provided herein further comprise providing osteogenic materials including osteogenic growth factors and/or growth factors to the bone. These materials may be factors found naturally in the body in bone or cartilage growth or maintenance or found associated with other cellular growth or maintenance, not found within the body. In some embodiments, the method further comprises sealing the cavity of the bone in which a device has been at least partially implanted using osteogenic materials including osteogenic growth factors and/or growth factors to the bone. In some embodiments the method comprises placing osteogenic materials including osteogenic growth factors and/or growth factors in the bone and/or on the bone to strengthen bone and/or to deflect, diffuse, or absorb forces (such as force from a fall or forces from normal use, as described herein. Examples of osteogenic materials may include, but are not limited to, FGF, angiogenic growth factors, LDL, minerals, osteoblasts causing osteogenesis, or factors used by osteoblasts, osteoprogenitor cells, or cells or factors used to restructure bone including osteoclasts, forms of remodeling or osteolysis. The methods provided herein may also comprise placing bone marrow or bone marrow-like cells in femur to enhance blood production.

Some embodiments comprise implanting a reinforcing material, as described herein.

FIG. 18 depicts an embodiment of a means for absorbing force comprising rods 6 and reinforcing material 74 implanted in a femur 32, and a pad 64 implanted substantially outside the femur 32 at the greater trochanter 46. In the embodiment of FIG. 18, the rods 6 have been inserted through the femoral neck and reinforcing material 74 has been implanted at about the locations of typical fractures, including, but not limited to at least one of the subcapital region, the cervical region, the basal region, and the pertrochaneric region of the femur. A pad 64, such as one of any embodiment described herein, has been implanted to interface with the greater trochanter. Such a device may be implanted by a method provided herein. For non-limiting example, the device may be implanted by creating a hole in the greater trochanter, and inserting wires (bundled and substantially parallel at the time of implantation) into the hole. The method may comprise affixing the wires to at least one of the wall of the hole and the distal end of the hole. The method may comprise placing the wires along at least one of the tensile force lines, and strain lines of a bone (wherein the tensile force and/or the strain results from at least one of normal use as described herein, ligamentary tension experienced during a fall, and the forces experienced in the bone during a fall). The wires may be unbundled to affix to the cavity walls, as the wires may be biased to a shape that is larger than the shape it has in its bundled form. The device of FIG. 18 may be capable of reinforcing and/or strengthening bone to withstand forces of normal use (as described herein), and forces of a fall.

The method of implanting the device depicted in FIG. 18 may further comprise injecting a reinforcing material as described herein into the hole or into the area where the scaffold has been deployed. The reinforcing material may harden in place. The reinforcing material may be implanted in typical fracture sites of the bone, for example, for the case of the femur, in at least one of the subcapital region, the cervical region, the basal region, and the pertrochaneric region. The reinforcing elements properties and variations described herein may be the reinforcing material.

Some embodiments of the methods provided herein comprise implanting a pad and implanting a rod 78 implanted along a load line, such as is depicted in FIG. 20. The rod 78 may also or alternatively be implanted generally along the bone axis. The method may also include implanting a scaffold 80 and reinforcing the bone with reinforcing material 74 as is shown in FIG. 20. The scaffold 80 may comprise at least one rod 78. Rods may include deflectable rods and/or coils which may compress longitudinally, expand longitudinally, or deflect laterally. The device of FIG. 20 may be capable of reinforcing and/or strengthening bone to withstand forces of normal use (as described herein), and forces of a fall.

The method may further comprise placing a pad at the greater trochanter to absorb, diffuse, and/or deflect forces of a fall on the greater trochanter.

The method may further comprise implanting a scaffold in the bone. The method may comprise deploying the scaffold at set areas corresponding to at least one of the subcapital region, the cervical region, the basal region, and the pertrochaneric region, which are typical fracture sites of the femur.

Provided herein is a kit for internally reinforcing a bone. In some embodiments, the kit comprises a set of implantable elements having a distal end and a proximal end for reinforcing a bone. Some embodiments of the kit comprises at least one device described herein. The kit may comprise a first implantable pad for securing the distal end of the rod within the bone. The kit may comprise a second implantable pad for securing the proximal end of the rod within the bone. The implantable element of the kit may comprise at least one of a solid rod, a collapsible rod, and a coil.

The kit may comprise a means for implanting at least one implantable element in the bone. The kits may include at least one tool for implantation of the device, devices, and/or implantable elements described herein. The means for implanting the set of rods in the bone may comprise at least one of a cutting tool and a bone drill, wherein the cutting tool and bone tool are adapted to prepare the bone for implantation of at least one of an implantable element, the first implantable pad, and the second implantable pad. In some embodiments, the means for implanting is capable of creating a cavity in bone. The cavity may be formed by, for non-limiting example, at least one of cutting, drilling, and compressing the bone, which may include trabecular and/or cortical bone. Other bone cavitation tools and methods of using the tools are contemplated herein. The cavity may be formed by drilling a hole in the bone, inserting a pressurizable bladder in the trabecular bone area of the bone, inflating the bladder in the hole, and compressing the trabecular bone generally toward the cortical bone by pressurizing the bladder. In another embodiment of the method, the cavity may be formed by drilling a hole in the bone, expanding the drilled hole by at least one of digging the trabecular bone out, and drilling additional areas of bone at an angle to the hole, using the hole drilled as an access point. Bone matter displaced by the cavity may and/or may not be removed from the bone. In some embodiments, the means for implanting the set of rods in the bone is capable of creating a cavity in the bone. In some embodiments, the bone is the femur, and wherein the means for implanting the set of rods in the bone is capable of creating a cavity in the bone about along the femur head axis running through the femur neck. In some embodiments, the kit comprises a guidewire. In some embodiments, the guidewire is flexible and/or rigid. In some embodiments, the kit comprises a system and/or a device for maintaining the existing vascular and neurological structure within the bone.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device comprising:
(a) an implantable pad,
wherein the pad is adapted to at least one of: absorb an exterior impact force on a bone, deflect an exterior impact force on a bone, diffuse an exterior impact force on a bone, strengthen a bone, and reinforce a bone.

2. The device of claim 1, comprising at least one of: a coil having a portion adapted to be implanted in the bone, a rod having a portion adapted to be implanted in the bone, and a reinforcing material adapted to be implanted in the bone,
wherein the pad and at least one of the implanted coil, the implanted rod, and the reinforcing material are adapted to cooperate in at least one of: absorbing an exterior impact force on the bone, deflecting an exterior impact force on the bone, diffusing an exterior impact force on the bone, strengthening the bone, and reinforcing the bone.

3. The device of claim 2, wherein at least one of the implanted coil and the implanted rod is adapted to substantially align with at least one of: a direction of load in the bone experienced in the bone during a fall, and a direction of load in the bone experienced in normal use.

4. The device of claim 3, wherein normal use includes at least one of walking, lifting, bending, sitting, running, leaning, and standing.

5. The device of claim 2, comprising a fastening element capable of fastening a distal end of at least one of the coil and the rod to at least one of trabecular bone and cortical bone of the bone.

6. The device of claim 1, wherein the bone is a femur.

7. A device comprising:
(a) a multiple-rod scaffold adapted to be implanted within a bone,
wherein the scaffold comprises a plurality of rods.

8. The device of claim 7, wherein at least one rod of the multiple-rod scaffold is adapted to be implanted substantially parallel relative to another rod of the scaffold, and wherein at least one rod of the multiple rod scaffold is adapted to be implanted at a displaced position relative to another rod of the scaffold.

9. The device of claim 7, wherein at least one rod of the multiple-rod scaffold is adapted to substantially align with at least one of: a direction of load in the bone experienced in the bone during a fall, and a direction of load in the bone experienced in normal use.

10. The device of claim 9, wherein normal use includes at least one of walking, lifting, bending, sitting, running, leaning, and standing.

11. The device of claim 7, wherein the multiple-rod scaffold is capable of at least one of: absorbing an exterior impact force on the bone, deflecting an exterior impact force on the bone, diffusing an exterior impact force on the bone, strengthening the bone, and reinforcing the bone.

12. The device of claim 7, wherein:
(a) at least one rod of the multiple-rod scaffold comprises an implant adapted to be implanted in the bone of a body at a first orientation and a first position relative to the bone, and
(b) at least one rod of the multiple-rod scaffold comprises a deployable element adapted to deploy in the body in at a second orientation and second position relative to the bone,
wherein at least one of the implant and the deployable element is capable of at least one of: absorbing an exterior impact force on the bone, deflecting an exterior impact force on the bone, diffusing an exterior impact force on the bone, strengthening the bone, and reinforcing the bone.

13. The device of claim 12, wherein the deployable element at least one of: unfolds, folds, expands radially from the implant, and expands distally from the implant.

14. The device of claim 12, wherein at least one of the implant and the deployable element of the implant is adapted to substantially align with at least one of: a direction of load in the bone experienced in a bone during a fall, and a direction of load in the bone experienced in normal use.

15. The device of claim 7, wherein the bone is a femur.

16. The device of claim 7, wherein the at least one rod in the multiple-rod scaffold is a coil.

17. The device of claim 7, further comprising a reinforcing material which cooperates with the multiple-rod scaffold to at least one of: absorb an exterior impact force on the bone, deflect an exterior impact force on the bone, diffuse an exterior impact force on the bone, strengthen the bone, and reinforce the bone.

18. A device comprising
(a) a fastening element capable of fastening to a bone at least one of:
a means for absorbing a force on the bone;
a means for deflecting a force on the bone;
a means for diffusing a force on the bone;
a means for reinforcing the bone to withstand a force on the bone; and
a means for strengthening the bone to withstand a force on the bone,
wherein the force results from at least one of a fall, and normal loading of the bone.

19. The device of claim 18, wherein at least one of the means for absorbing, the means for deflecting, the means for diffusing, the means for reinforcing, and the means for strengthening comprises at least one of a pad, a rod, a collapsible rod, and a coil.

20. The device of claim 18, wherein at least one of the means for absorbing, the means for deflecting, the means for diffusing, the means for reinforcing, and the means for strengthening comprises a material having at least about the same yield strength than at least one of trabecular bone and cortical bone.

21. The device of claim 18, wherein the means for deflecting the force on the bone deflects the force in a direction whereby the force is directed to at least one of:
a portion of the bone that can withstand the force without fracturing the bone,
another body part that can withstand the force without fracturing the bone, and
a material external to the body that can withstand the force without fracturing the bone.

22. The device of claim 18, wherein at least one of the means for reinforcing and the means for strengthening is adapted to be implanted in at least one of: a location in the bone of high stress during at least one of normal use and a fall, and a location in the bone of high strain during at least one of normal use and a fall.

23. The device of claim 22, wherein normal use includes at least one of walking, lifting, bending, sitting, running, leaning, and standing.

24. The device of claim 18, wherein the means for maintaining in position comprises a fastening element.

25. The device of claim 18, wherein the bone is a femur.

26. A method for reducing the risk of fracturing a bone of a patient comprising:
implanting a pad adapted to interface with the bone,
wherein the pad is adapted to at least one of: absorb an exterior impact force on the bone, deflect an exterior impact force on the bone, diffuse an exterior impact force on the bone, strengthen the bone, and reinforce the bone.

27. The method of claim 26 comprising at least one of:
(a) delivering at least a portion of a coil within the bone;
(b) delivering at least a portion of a rod within the bone; and
(c) delivering a reinforcing material within the bone,
wherein the pad and at least one of the coil, the rod, and the reinforcing material are adapted to cooperate in absorbing an exterior impact force on the bone, deflecting an exterior impact force on the bone, diffusing an exterior impact force on the bone, strengthening the bone, and/or reinforcing the bone.

28. The method of claim 26, wherein the force is from normal loading of the bone.

29. The method of claim 26, wherein the bone is a femur.

30. A method for reducing the risk of fracturing a bone of a patient comprising:
(a) implanting a multiple-rod scaffold within the bone, wherein the implanting comprises:
(i) orienting each rod substantially parallel to each other rod, and
(ii) positioning each rod at a displaced position in the bone relative to each other rod.

31. The method of claim 30, wherein the bone is a femur.

32. A method for reducing the risk of fracturing a bone of a patient comprising:
(a) implanting an implant in the bone at a first orientation and a first position relative to the bone, wherein the implant comprises an deployable element adapted to deploy within the bone at a second orientation and second position relative to the bone, and
(b) deploying the deployable element at the second orientation and second position relative to the bone.

33. The method of claim 32, wherein the bone is a femur.

34. A kit comprising:
(a) a set of implantable elements having a distal end and a proximal end for reinforcing a bone,
(b) a first implantable pad for securing the distal end of the rod within the bone,
(c) an means for implanting at least one implantable element in the bone, and
(d) a second implantable pad for securing the proximal end of the rod within the bone.

35. The kit of claim 34, wherein the implantable element comprises at least one of a solid rod, a collapsible rod, and a coil.

36. The kit of claim 34, wherein the bone is a femur.

37. The kit of claim 34, further comprising a reinforcing material.
